(19) United States
(12) Patent Application Publication
(10) Pub. No.: US 2024/0287481 A1
(43) Pub. Date: Aug. 29, 2024

(54) POLYMERASES FOR EFFICIENT INCORPORATION OF NUCLEOTIDES WITH 3'-PHOSPHATE AND OTHER 3'-TERMINATORS

(71) Applicant: MGI Tech Co., Ltd., Shenzhen (CN)

(72) Inventors: Radoje T. Drmanac, Los Altos Hills, CA (US); Matthew J. Callow, Redwood City, CA (US); Snezana Drmanac, Los Altos Hills, CA (US)

(21) Appl. No.: 18/551,031

(22) PCT Filed: Mar. 17, 2022
(86) PCT No.: PCT/CN2022/081438

§ 371 Date: Sep. 18, 2023

Related U.S. Application Data
(60) Provisional application No. 63/163,629, filed on Mar. 19, 2021.

(51) Int. Cl. C12N 9/12; C12P 19/34; C12Q 1/6806

(52) U.S. Cl. CPC C12N 9/1252; C12P 19/34; C12Q 1/6806; C12Y 207/07007

(57) ABSTRACT

Provides are variant family A polymerases that incorporate 3′-blocked nucleotides into a DNA extension product, a kit comprising such polymerases and methods of using the polymerases in DNA extension reactions, e.g., sequencing reactions.

### CROSS-REFERENCE TO RELATED APPLICATIONS

This application claims priority benefit of Provisional Application No. 63/163,629, filed Mar. 19, 2021, which is incorporated by reference for all purposes.

### REFERENCE TO AN ELECTRONIC SEQUENCE LISTING

The contents of the electronic sequence listing (106340-1390100-5101-US SL; Size: 65,812 bytes; and Date of Creation: Dec. 21, 2023) is herein incorporated by reference in its entirety.

### BACKGROUND OF THE INVENTION

The incorporation of 3′-phosphate-modified reversible nucleotides in a templated DNA strand for the purpose of sequence determination was previously suggested. Tsien et al., WO1991006678A1 reported the use of the 3′ phosphatase activity of T4 polynucleotide kinase as a mechanism for conversion of the phosphate blocking group to an extendable 3′-hydroxyl group. Although the use of a DNA polymerase for chain extension was suggested, no information for suitable polymerases that could incorporate a 3′ phosphate was provided.

Reports of the possibility of incorporating a 3′ phosphate blocking group of an extending non-templated DNA strand with reversal to a 3′ OH group for further extension have also been proposed. U.S. Pat. No. 5,990,300 described the nucleotide addition for the purposes of chain synthesis rather than template sequence determination and suggested the use of deoxynucleotidyl terminal transferase for addition of terminators. Suggested incorporation conditions included the use of manganese and 100 μM nucleotide concentrations.

Zhou et al. (U.S. Pat. No. 9,650,406) further reported attempts to utilize a 3′ monophosphate as a blocking group as having been unsuccessful; and further noted incorporation of reversible terminators comprising a phosphate diester group at the 3′ oxygen of the sugar moiety. They suggested mutant forms of 9° N-7(exo-) polymerase such as Therminator™ (New England Biolabs), exo-mutant of KOD DNA polymerase and enhanced DNA polymerase, or EDP (WO 2005/024010) as suitable polymerases for incorporation.

Fa et al. 2004 (Fa et al., J. Am. Chem. Soc. 126, 1748-175, 2004) reported the use of phage display with co-localization of the expressed enzyme and an oligonucleotide primer substrate to identify Taq polymerase Stoffel fragment mutants with the ability to incorporate 2′-O-methyl ribonucleoside triphosphates. They performed random mutagenesis at positions Ile614, Glu615, Phe667, Tyr671, Asn750, and Gln754, because of their reported association with magnesium and triphosphate nucleotide binding. In a separate library Arg573, Met747, Gln754, Val783, His784, and Glu786 were selected because of their reported association with the primer terminus binding. They selected multiple candidates and identified one, SFM19, with mutations Ile614Glu and Glu615Gly, as the strongest candidate for incorporation of 2′-O-methyl ribonucleoside triphosphates. The amino acids 614 and 615 form part of the motif A conserved in polymerases and, as in homologous regions of polymerases such as Klenow, are believed to form part of the steric gate restricting the incorporation of 2′-OH nucleotides. Mutations at Glu 615, or the homologous Glu710 in Klenow, had previously been shown to facilitate the incorporation of rNTPs (Astatke, et al., Proc. Natl. Acad. Sci. USA 95:3402-3407; 1998; Patel et al., J. Biol. Chem. 275:40266-40272, 2000).). It was also suggested that mutation of Taq 615 to accommodate the substituted nucleotide then required the 614 mutation to maintain activity (Fa et al., supra).

Chen et al. (Chen et al., Nat Chem. 8:556-562, 2016) performed up to 4 rounds of selection on the SFM19 substrate to modify amino acids thought to be important in permitting continued incorporation of C2′-OMe modified nucleotides on C2′-OMe modified primers. They identified several variants of SFM19 described as SFM4-3, SFM4-6 and SFM4-9. Each variant was reported to transcribe fully C2′-OMe-modified oligonucleotides but the greatest activity was observed with SFM4-6. An analysis of one of the mutants (SFM4-3) suggested that the ability to amplify the C2′-modified oligonucleotides resulted from stabilizing the interaction between the fingers and thumb domains which favors the formation of the catalytically active closed complex. Ong et al (Ong, et al., J Mol Biol. 361:537-550, 2006) reported a mutant AA40 (E602V, A608V, I614M, E615G) with similar properties of incorporating 2′ substituted nucleotides such as 2′-fluoro- and 2′-azido-derivatives but noted substituents such as NH2 or OCH3 were only poorly extended.

Ghadessy et al. (Ghadessy et al., Proc. Natl. Acad. Sci. USA 98, 4552-4557, 2001) identified mutants with increased heparin tolerance that shared some of the same mutations as 4-3 and 4-6. H15 (K225E, E388V, K540R, D578G, N583S, M747R) was a variant of Taq polymerase that remained fully functional in PCR at up to 130 times the inhibitory concentration of heparin.

Davis et al. (WO2001014568) reported Taq polymerase with substitution at E681 with improved salt tolerance.

Brandis et al. (U. S. Pat. Appln. Publ. No. 20150232822) reported D655 and E681 as two of several amino acids that could be substituted for improved labeled nucleotide acceptance.

The SFM19 polymerase was selected by Guo for incorporation of 2′ phosphate modified nucleotides in 2016 (Guo, thesis, Columbia University 2016). Guo reported the difficulty in identifying polymerases that can incorporate 3′ blocked nucleotides but describes the use of SFM19 for the incorporation of 2′-phosphate modified nucleotides with the associated benefit of inhibiting further extension after incorporation. Extension could be continued with enzymatic removal of the 2′ phosphate group. However, it was not clear if continued cycles of incorporation and cleavage could be maintained with SFM19 and the generation of 2′-OH residues on the extending primer.

Taq polymerase residue 667 and T7 DNA polymerase 762 are considered important residues for the incorporation of dideoxy nucleotides with increased acceptance of these modified nucleotides when the phenylalanine is substituted with tyrosine (Tabor, et al., Proc Natl Acad Sci USA 92:6339-6343, 1995).

The wild-type amino acid at Taq position 614 is the hydrophobic isoleucine but in sfm19 it is substituted with the negatively charged glutamic acid. Patel et al. (Patel, et al, J Biol Chem. 276:5044-5051, 2001) reported conversion of the I614 to the positively charged lysine increased the ability to extend a primer on a damaged substrate but may permit decreased fidelity.

Chen et al. (F. Chen, et al, Proc. Natl. Acad. Sci. USA 107:1948-1953, 2010) used a process of Reconstructing Evolutionary Adaptive Paths (REAP) to identify critical amino acids that could facilitate the incorporation of 3′-ONH2 modified nucleotides. Active variants for acceptance of the modified nucleotide included substitutions of L616A and F667Y. L616A alone appeared to give the most improved acceptance of the 3′-ONH2 compared with I614G and F667Y, but both I614G and F667Y demonstrated some improved acceptance of the modified nucleotide compared with wild-type. They also demonstrated the ability of the L616A modification to improve acceptance of dideoxy nucleotides.

Many polymerases that incorporate bulky 3′-groups (BG9, KOD, Taq475, Sequenase) have not been demonstrated to efficiently incorporate a 3′ phosphate blocking group. SFm19, which as noted above, is known to incorporate 2′ phosphate nucleotides also has not been demonstrated to incorporate 3′ phosphate nucleotides.

Thermosequenase has an F667Y mutation that allows it to incorporate ddNTPs wherein a Tyrosine (Y) provides a —OH group (missing at the 3′ position in ddNTPs) for Mg++ to bind. Taq475 has no F667Y mutation and still incorporates 3′-O—NH2 but does not incorporate 3′-phosphate nucleotides.

SFm19, which incorporates 2′-phosphate nucleotides has two amino acid mutations in the A motif (positions 614 and 615). However, the variant polymerase 9° N (also referred to as BG9) with 3 mutations in the corresponding A-motif (408, 409, 410) does not incorporate 2′-phosphate.

### BRIEF SUMMARY

This section provides a summary of certain aspects of the disclosure. The invention is not limited to embodiments summarized in this section.

The present disclosure provides variant, engineered Family A polymerases, e.g. Taq polymerase, having mutations that permit template-directed incorporation of 3′-phosphate nucleotides and 3′-O-blocked nucleotides, such as 3′-O-nitrobenzyl (NB)-modified nucleotides, into an extension product of a primer. In one aspect, the disclosure features an engineered DNA polymerase comprising a polypeptide sequence comprising a substitution at three, four, or five, or more of positions 610, 611, 612, 613, 614, 615, 616, and 617, as determined with reference to SEQ ID NO: 1; and wherein the polypeptide sequence has at least 90% identity to SEQ ID NO:1 and incorporates a 3′-blocked nucleotide into a nucleic acid chain. In some embodiments, the polypeptide sequence comprises a substitution at three or more of positions 613, 614, 615, 616, or 617. In some embodiments, two positions are substituted with G; and a third position is substituted with S or T. In some embodiments the polypeptide sequence comprises an A or a G at three or more of positions 613, 614, 615, 616, or 617. In some embodiments, each of positions 614, 615, and 616 comprises a substitution relative to SEQ ID NO:1. In some embodiments, the substitution at position 614 is E, A, D, S, or G; the substitutions at position 615 is A, G, or S; or the substitution at position 616 is A, G, or S. In some embodiments, the substitution at position 614 is E or S; the substitutions at position 615 is G; and the substitution at position 616 is A. In some embodiments, the substitution at position 614 is S. In some embodiments position 614 is E or S; position 615 is G or E; and position 616 is A. In some embodiments the engineered polymerase, further comprising a substitution at position 667, such as Y, as determined with reference to SEQ ID NO:1. In some embodiments, the engineered DNA polymerase further comprises a substitution at one, two, three, or four of positions 655, 657, 681, 742, and 747, as determined with reference to SEQ ID NO:1. In some embodiments, the engineered DNA polymerase further comprises a substitution at each of positions 655, 657, 681, 742, and 747, as determined with reference to SEQ ID NO:1. In some embodiments, the substitution at position 655 is N; the substitution at position 657 is M; the substitution at position 681 is K; the substitution at position 742 is Q or N; or the substitution at position 747 is R. In some embodiments, the substitution at position 655 is N; the substitution at position 657 is M; the substitution at position 681 is K; the substitution at position 742 is Q or N; and the substitution at position 747 is R. In some embodiments, the polymerase comprises E, A, D, S or G at position 614; G at position 615; A or G at position 616; and Y at position 667. In some embodiments, the polymerase further comprises an N at position 655; an M at position 657; a K at position 681; a Q or N at position 742; and a T at position 747.

In a further aspect, provided herein is an engineered DNA polymerase comprising a polypeptide sequence comprising substitutions at two of positions 613, 614, 615, 616, and 617; and at position 667, as determined with reference to SEQ ID NO:1; wherein the polypeptide sequence has at least 90% identity to SEQ ID NO:1 and incorporates a 3′-blocked nucleotide into a nucleic acid chain. In some embodiments, the substitution at each of the two positions is A, G, or S. In some embodiments, the polypeptide sequence comprises substitution at positions 614 and 615; as determined with reference to SEQ ID NO: 1. In some embodiments, the substitution at positions 614 is E, A, D, S, or G; the substitution at position 615 is G; or the substitution at position 667 is Y. In some embodiments, the substitution at positions 614 is E, A, D, S, or G; the substitution at position 615 is G; and the substitution at position 667 is Y. In some embodiments, the residue at position 614 is A or G. In some embodiments, the polypeptide sequence comprises a substitution at position 615 and 616, as determined with reference to SEQ ID NO:1. In some embodiments the substitution at each of positions 615 and 616 is S, G, or A. In some embodiments, the substitution at each of positions 615 and 616 is G or A. In some embodiments, the engineered DNA polymerase further comprises a substitution at each of positions 655, 657, 681, 742, and 747. In some embodiments, the substitution at position 655 is N; the substitution at position 657 is M; the substitution at position 681 is K; the substitution at position 742 is Q or N; or the substitution at position 747 is R. In some embodiments, the substitution at position 655 is N; the substitution at position 657 is M; the substitution at position 681 is K; the substitution at position 742 is Q or N; and the substitution at position 747 is R.

In a further aspect, the disclosure features an engineered family A polymerase comprising a set of two or more substitutions, relative to the wild-type family A polymerase sequence that together provide a larger space for accommodating a 3′-phosphate or a larger 3′-blocking group wherein the substitutions are each selected from the group consisting of A, G, S, T, and C. In some embodiments the family A polymerase further comprises a Y at the position of the family A polymerase that corresponds to position 667 of Taq polymerase. In some embodiments, the family A polymerase comprises at least two positions in motif A in which G or A is substituted for the wildtype residue; and at least one position in motif A comprises S or T. In some embodiments, a wildtype L or I residue of motif A is substituted with E, S, or T in the engineered family A polymerase.

In another aspect, the disclosure provides a method of incorporating a nucleotide comprising a 3′-blocking substituent into a DNA molecule, the method comprising incubating a polymerase as described herein, e.g., in the paragraphs in this section, in a reaction mixture comprising a template nucleic acid, a primer, the nucleotide with the 3′-blocking group; and reagents to extend the primer, wherein the nucleotide comprising the 3′-blocking group is complementary to a base in the template nucleic acid and is incorporated into the DNA molecule. In some embodiments, the nucleotide is labeled with a detectable label.

In an additional aspect, the disclosure provides a method of incorporating a first nucleotide comprising a reversible 3′-blocking substituent into a DNA molecule, the method comprising incubating a polymerase as described herein, e.g., in the paragraphs in this section, in a reaction mixture comprising a template nucleic acid, a primer, the nucleotide with the 3′-blocking group; and reagents to extend the primer, wherein the nucleotide comprising the 3′-blocking group is complementary to a base in the template nucleic acid and is incorporated into the extension molecule extended from the primer; detecting the nucleotide comprising the 3′-blocking group incorporated into the extension molecule; and removing the 3′ blocking substituent. In some embodiments, the nucleotide is labeled with a detectable label. In some embodiments, the method further comprises incubating the extension molecule in the reaction mixture with a second nucleotide comprising a reversible blocking substituent; wherein the second nucleotide comprising the 3′-blocking group is complementary to a base in the template nucleic acid and is incorporated into the extension molecule.

In a further aspect, the disclosure provides a kit comprising a polymerase as described herein, e.g., in the paragraphs in this section. In some embodiments, the kit further comprises one or more 3′-blocked nucleotides.

In other aspects, the disclosure polynucleotide comprising a nucleic acid sequence encoding a polymerase as described herein, e.g., in the paragraphs in this section; and a host cell comprising such a polynucleotide.

### DESCRIPTION OF DRAWINGS

FIG. 1. DNB intensity plots of one cycle of sequencing. DNBs prepared from an E. coli genomic library were loaded onto an MGI-SEQ 2000 sequencer flowcell followed by primer hybridization. 3′ phosphate blocked nucleotides were incorporated with SFr52 polymerase by reacting a mixture of 4 nucleotides (dATP-3P, dCTP-3P, dGTP-3P, dTTP-3P) with the DNBs. After washing away excess reaction components, the DNBs were then reacted with a mixture of four fluorescently labeled antibodies, with each antibody type labeled with a different fluorophore. The antibody clones were A-18B10, C-2F7, G-6B9, and T-12D6. The antibodies were allowed to bind for 4 min, before washing excess unbound antibodies and imaging. The DNB intensities from one imaging field are represented.

FIG. 2A. Intensity change over consecutive sequencing cycles on the MGI-SEQ2000 sequencer. Background subtracted, phase corrected and spectral cross-talk corrected intensities are shown. For each imaging channel (corresponding to each base) an average intensity of DNBs with highest intensities in that channel are depicted. The combined data of 6 pre-selected fields is shown. Upperline (X symbol), T; second line (diamond symbol), A; third line (square symbol), G; fourth line (circle symbol), C

FIG. 2B. Positional discordance of the sequenced DNBs relative to a reference human sequence over 20 sequencing cycles on the MGI-SEQ2000 sequencer. The combined data of 6 pre-selected fields is shown.

### DETAILED DESCRIPTION

### Terminology

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “an agent” includes reference to one or more agents known to those skilled in the art, and so forth.

As used herein, a “native” nucleotide refers to a naturally occurring nucleotide that does not include an exogenous label (e.g., a fluorescent dye, or other label) or chemical modification, such as a blocking substituent. As used herein, a “nucleotide analog” has one or more modifications, such as chemical moieties, which replace, remove and/or modify any of the components (e.g., nitrogenous base, five-carbon sugar, or phosphate group(s)) of a native nucleotide.

Nucleotide analogs may be either incorporable or non-incorporable by a polymerase in a nucleic acid polymerization reaction. In some embodiments, the 3′-OH group of a nucleotide analog is modified with a moiety, which may be a reversible or irreversible terminator of polymerase extension. The base of a nucleotide may be any of adenine, cytosine, guanine, thymine, or uracil, or analogs thereof. Optionally, a nucleotide has an inosine, xanthine, hypoxanthine, isocytosine, isoguanine, nitropyrrole (including 3-nitropyrrole) or nitroindole (including 5-nitroindole) base. Nucleotides may include, but are not limited to, ATP, UTP, CTP, GTP, ADP, UDP, CDP, GDP, AMP, UMP, CMP, GMP, dATP, dTTP, dUTP, dCTP, dGTP, dADP, dTDP, dCDP, dGDP, dAMP, dTMP, dCMP, and dGMP. Nucleotides may also contain terminating inhibitors of DNA polymerase, dideoxynucleotides or 2′, 3′ dideoxynucleotides, which are abbreviated as ddNTPs (ddGTP, ddATP, ddTTP, ddUTP and ddCTP).

As used herein, a “blocking moiety,” when used with reference to a nucleotide analog, is a part of the nucleotide that inhibits or prevents the nucleotide from forming a covalent linkage to a second nucleotide (e.g., via the 3′-OH of a primer nucleotide) during the incorporation step of a nucleic acid polymerization reaction. The blocking moiety of a “reversible terminator” nucleotide can be modified or removed from the nucleotide analog to allow for nucleotide incorporation. Such a blocking moiety is referred to herein as a “reversible terminator moiety.” Equivalent or alternative terms include “blocking group”, “reversible blocking group”, “protecting group”, “cleavable protecting group”, and the like.

Exemplary reversible terminator moieties are set forth in WO2016/065248 and US2018/0223358, which are incorporated by reference.

The term “3′-blocking” nucleotide as used herein refers to a nucleotide having a purine or pyrimidine ribose or deoxyribose sugar moiety in which the naturally occurring free 3′ sugar hydroxyl is replaced with a blocking group that does not permit an additional nucleotide to be incorporated for extension of a nucleotide chain. In some embodiments, the 3′ hydroxyl is replaced with a 3′ phosphate group. In some embodiments, e.g., for sequencing, the 3′ blocking group may then be removed to allow incorporation of a further nucleotide as desired. In the present application, the terms 3′-blocking nucleotide” and “3′-terminator nucleotide” are used interchangeably.

A “3′-phosphate” nucleotide as used herein refers to a nucleotide having a purine or pyrimidine base and a ribose or deoxyribose sugar moiety in which the 3′ sugar hydroxyl is modified to replace the naturally occurring 3′ hydroxyl with a phosphate.

The term “large 3′ blocking substituent” as used herein refers to a substituent group at the 3′ sugar hydroxyl which is larger in size than the naturally occurring 3′ hydroxyl group. For illustration and not limitation, nucleotides that have been modified at the 3′ sugar hydroxyl such that the substituent is larger in size than the naturally occurring 3′ hydroxyl group are also disclosed in WO 2004/018497 and WO2018/148727.

Herein, “incorporation” means joining of the modified nucleotide to the free 3′ hydroxyl group of a second nucleotide via formation of a phosphodiester linkage with the 5′ phosphate group of the modified nucleotide. The second nucleotide to which the modified nucleotide is joined will typically occur at the 3′ end of a polynucleotide chain.

As used herein, a “template nucleic acid” is a nucleic acid to be acted upon (e.g. amplified, detected or sequenced) using a method or composition disclosed herein.

Amino acids can be identified using a three-letter code or a one letter code in accordance with the conventions in the art.

| | 1 letter | |
|---|---|---|
| Abbreviation | abbreviation | Amino acid name |
| Ala | A | Alanine |
| Arg | R | Arginine |
| Asn | N | Asparagine |
| Asp | D | Aspartic acid |
| Cys | C | Cysteine |
| Gln | Q | Glutamine |
| Glu | E | Glutamic acid |
| Gly | G | Glycine |
| His | H | Histidine |
| Ile | I | Isoleucine |
| Leu | L | Leucine |
| Lys | K | Lysine |
| Met | M | Methionine |
| Phe | F | Phenylalanine |
| Pro | P | Proline |
| Pyl | O | Pyrrolysine |
| Ser | S | Serine |
| Sec | U | Selenocysteine |
| Thr | T | Threonine |
| Trp | W | Tryptophan |
| Tyr | Y | Tyrosine |
| Val | V | Valine |
| Asx | B | Aspartic acid or Asparagine |
| Glx | Z | Glutamic acid or Glutamine |
| Xaa | X | Any amino acid |
| Xle | J | Leucine or Isoleucine |

A “conservative” substitution as used herein refers to a substitution of an amino acid such that charge, polarity, hydropathy (hydrophobic, neutral, or hydrophilic), and/or size of the side group chain is maintained. Illustrative sets of amino acids that may be substituted for one another include (i) positively-charged amino acids Lys and Arg; and His at pH of about 6; (ii) negatively charged amino acids Glu and Asp; (iii) aromatic amino acids Phe, Tyr and Trp; (iv) aliphatic hydrophobic amino acids Ala, Val, Leu and Ile; and hydrophobic amino acid Met; (v) non-polar amino acids Ala, Val, Leu, Ile, Pro, Phe, Trp, and Met; (vi) small polar uncharged amino acids such as Ser, Thr, and Asn; (vii) neutral hydrophilic amino acids Cys, Ser, Thr, Asn, Gln; (viii) small hydrophobic or neutral amino acids Gly, Ala, and Pro; (ix) amide-comprising amino acids Asn and Gln; and (x) branched amino acids Thr, Val, and Ile. In some embodiments, a conservative substitution may be based on size, for example, a small amino acid may be substituted with another small amino acid, such as Gly or Ala. In some embodiments, a hydroxyl-containing amino acid (Ser, Thr, or Tyr) may be substituted with an alternative hydroxyl-containing amino acid. Reference to the charge of an amino acid in this paragraph refers to the charge at pH 6-7.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. The polypeptide that is aligned to the reference sequence need not be the same length as the reference sequence.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Nat. Acad. Sci. USA 89:10915 (1989)). For purposes of this application, amino acid sequence identity is determined using BLASTP with default parameters.

As used herein, the terms alkyl, alkenyl, and alkynyl include straight- and branched-chain monovalent substituents. Examples include methyl, ethyl, isobutyl, 3-butynyl, and the like. Ranges of these groups useful with the compounds and methods described herein include C₁-C₂₀ alkyl, C₂-C₂₀ alkenyl, and C₂-C₂₀ alkynyl. Additional ranges of these groups useful with the compounds and methods described herein include C₁-C₁₂ alkyl, C₂-C₁₂ alkenyl, C₂-C₁₂ alkynyl, C₁-C₆ alkyl, C₂-C₆ alkenyl, C₂-C₆ alkynyl, C₁-C₄ alkyl, C₂-C₄ alkenyl, and C₂-C₄ alkynyl.

Aryl molecules include, for example, cyclic hydrocarbons that incorporate one or more planar sets of, typically, six carbon atoms that are connected by delocalized electrons numbering the same as if they consisted of alternating single and double covalent bonds. An example of an aryl molecule is benzene. Heteroaryl molecules include substitutions along their main cyclic chain of atoms such as O, N, or S. When heteroatoms are introduced, a set of five atoms, e.g., four carbon and a heteroatom, can create an aromatic system. Examples of heteroaryl molecules include furan, pyrrole, thiophene, imadazole, oxazole, pyridine, and pyrazine. Aryl and heteroaryl molecules can also include additional fused rings, for example, benzofuran, indole, benzothiophene, naphthalene, anthracene, and quinoline. The aryl and heteroaryl molecules can be attached at any position on the ring, unless otherwise noted.

The term alkoxy as used herein is an alkyl group bound through a single, terminal ether linkage. Likewise, the term aryloxy as used herein is an aryl group bound through a single, terminal ether linkage.

The terms amine or amino as used herein are represented by the formula —NZ¹Z², where Z¹ and Z² can each be a substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above.

The alkoxy, aryloxy, amino, alkyl, alkenyl, alkynyl, and aryl molecules used herein can be substituted or unsubstituted. As used herein, the term substituted includes the addition of a substitution group to a position attached to the main chain of the alkoxy, aryloxy, amino, alkyl, alkenyl, alkynyl, and aryl, e.g., the replacement of a hydrogen by a substitution group. Examples of substitution groups include, but are not limited to, hydroxyl, halogen (e.g., F, Br, Cl, or I), and carboxyl groups. Conversely, as used herein, the term unsubstituted indicates the alkoxy, aryloxy, amino, alkyl, alkenyl, alkynyl, or aryl has a full complement of hydrogens, i.e., commensurate with its saturation level, with no substitutions, e.g., linear decane (—(CH₂)₉—C

### Polymerase Variants

The present disclosure features family A polymerase variants, e.g., Taq polymerase variants, that can incorporate blocking 3′-phosphate nucleotides or other 3′-terminator nucleotides in which the blocking substituent is similar in size or larger than phosphate. Members of polymerase family A, which include bacterial and bacteriophage polymerases, are classified based on amino acid sequence homology to Escherichia coli polymerase I (Braithwaite and Ito, Nuc. Acids. Res. 21:787-802, 1993). The family A polymerase family is thus also known as the pol I family. Type A polymerases include E. coli pol I, Thermus aquaticus DNA pol I (Taq polymerase), Thermus flavus DNA pol I, Streptococcus pneumoniae DNA pol I, Bacillus stearothermophilus pol I, phage polymerase T5, phage polymerase T7, mitochondrial DNA polymerase pol gamma, and others.

Three motifs, A, B and C are conserved across all DNA polymerases, with motifs A and C also seen in RNA polymerases. In some embodiments, an engineered family A polymerase in accordance with the disclosure can incorporate 3′-blocked nucleotides has at least two mutations relative to the wildtype family A polymerase sequence. In some embodiments, the blocking group has 3-5 or 4-15 non-H atoms. In some embodiments, a family A polymerase of the present disclosure has at least 2, or at least 3, mutations in motif A that creates a larger polymerase pocket and/or positions a hydroxyl group closer to promote interaction with Mg+². In some embodiments, the engineered family A polymerase further comprises Y at a position that corresponds to position 667 of SEQ ID NO:1. In some embodiments, an engineered family A polymerase comprises a G, A, S, T, or C substitution for a wildtype residue in motif A or residues behind the region of motif A close to the 3′ end of the primer (e.g., the region of motif A of the family A polymerase corresponding to positions 614-616 of SEQ ID NO:1). In some embodiments, an engineered family A polymerase comprises three or more substitutions, compared to the wildtype family A polymerase sequence, in motif A. In some embodiments, a G is substituted for the native residue in order to create a maximal pocket opening. In some embodiments, at least two positions of motif A are G or A and at least one position is S. In alternative embodiments, a wildtype residue of motif A is substituted with T. In other embodiments, multiple positions, e.g., two or more positions of motif A, may have an S, G, or A. In some embodiments, three or more positions of motif A are S, G or A. In some embodiments, a wildtype L or I or similar residue of motif A of a family A polymerase is substituted with E, S, or T, or a similar residue, e.g., to provide a less hydrophobic and more negatively charged or polar neutral environment. In some embodiments, the engineered family A polymerase may have one or more mutations in the primer binding domain.

In naturally-occurring polymerases, motif A contains a conserved aspartate at the junction of a beta-strand and an alpha-helix in the palm subdomain. In the case of Taq polymerase, this catalytic aspartate corresponds to residue D610, as numbered using a full-length Taq polymerase sequence. Residue D610 is reported to interact with the incoming dNTP and stabilize the transition state that leads to phosphodiester bond formation. Motif A of Taq polymerase corresponds to the 13 residues from positions 605-617, with the most conserved region of the motif corresponding to residues 610 to 617.

In some embodiments, a polymerase as described herein, which incorporates 3′-blocked nucleotides, is a biologically active fragment. The sequence of a 540-amino acid fragment (Stoffel fragment, also referred to in the art as KlenTaq fragment) of Taq polymerase is provided in SEQ ID NO:1. The fragment lacks the N-terminal 292 amino acids. For purposes of this application, numbering of residues employs the numbering of the positions in full-length Taq polymerase. SEQ ID NO:1 shows residues 293 through 832 of Taq polymerase. Thus, the first position of the sequence of SEQ ID NO:1 is designated as position 293 in the present application. A highly conserved portion of motif A of Taq polymerase from position 610-617 is underlined. Residue 667 is also underlined.

(SEQ ID NO: 1)
ALEEAPWPPPEGAFVGFVLSRKEPMWADLLALAAARGGRVHRAPEPYKALRDLKEARGLLAK
DLSVLALREGLGLPPGDDPMLLAYLLDPSNTTPEGVARRYGGEWTEEAGERAALSERLFANL
WGRLEGEERLLWLYREVERPLSAVLAHMEATGVRLDVAYLRALSLEVAEEIARLEAEVFRLA
GHPFNLNSRDQLERVLFDELGLPAIGKTEKTGKRSTSAAVLEALREAHPIVEKILQYRELTK
LKSTYIDPLPDLIHPRTGRLHTRFNQTATATGRLSSSDPNLQNIPVRTPLGQRIRRAFIAEE
GWLLVALDYSQIELRVLAHLSGDENLIRVFQEGRDIHTETASWMFGVPREAVDPLMRRAAKT
INFGVLYGMSAHRLSQELAIPYEEAQAFIERYFQSFPKVRAWIEKTLEEGRRRGYVETLFGR
RRYVPDLEARVKSVREAAERMAFNMPVQGTAADLMKLAMVKLFPRLEEMGARMLLQVHDELV
LEAPKERAEAVARLAKEVMEGVYPLAVPLEVEVGIGEDWLSAKE

In some embodiments, an engineered polymerase in accordance with the disclosure that incorporates 3′-blocked nucleotides, e.g., an engineered Taq polymerase, has at least two mutations in motif A and a mutation at position 667. In some embodiments, the two or more mutations are at positions 613, 614, 615, 616, or 617. In some embodiments, the substitution at two or more of positions 613, 614, 615, 616, or 617 is G, A, S, T, or C. In some embodiments, the engineered Taq polymerase has at least 3 mutations in motif A that create a larger polymerase pocket and/or position a hydroxyl group to promote interaction with Mg+², e.g., substitutions at three or more of positions 613, 614, 615, 616, and/or 617. In some embodiments, the polymerase comprises S or T at one or more of positions 613, 614, 615, 616, or 617. In some embodiments, a G is substituted for the native residue in order to create a maximal pocket opening. In some embodiments, at least two of residues 613, 614, 615, 616, or 617 are G or A and at least one position is S. In some embodiments, position 614 is substituted with an S. In alternative embodiments, position 613, 614, 615 616, or 617 is T. For example, in some embodiments, position 614 is substituted with a threonine. In some embodiments, position 614 is D or T. In some embodiments, the residue at position 616 is replaced with an A or G. In other embodiments, multiple positions, e.g., two or more of positions 613, 614, 615, 616, or 617 are S, G, or A. In some embodiments, three or more of positions 613, 614, 615, 616, or 617 are S, G or A. In some embodiments, position 613 and/or position 617 is S or T. In some embodiments, position 614 is S, position 615 is G, and position 616 is A. In some embodiments, position 614 is T, position 615 is G, and position 616 is A. In some embodiments, position 617 is F or K. In some embodiments, the engineered polymerase comprises a substitution at position 667, e.g., a Y substituted for F at position 667; and/or one or more mutations in the primer binding domain, e.g., at one or more of positions 655, 657, 681, 742, and 747, as determined with reference to SEQ ID NO: 1. In some embodiments, the polymerase comprises N at position 655; M at position 657, K at position 681, Q or N at position 742, or R at position 747. In some embodiments, polymerase comprises D or N at position 655; M or L at position 657; E or K at position 681; E, N, or Q at position 742; and M or R at position 747.

In some embodiments, an engineered polymerase that incorporates a 3′-blocked nucleotide comprises a substitution at 614, 615, and 616; and a substitution at position 667, relative to SEQ ID NO: 1. In some embodiments, one, two, or all of positions 614, 615, and 616 are A or a G. In some embodiments, two of positions 614, 615, and 616 are A or G and the third positions is S. In some embodiments, the residue at position 614 is S or T. In some embodiments, the residue at position 614 is S or T, the residue at position 615 is G or A, and the residue at position 616 is G or A. In some embodiments, the residue at position 614 is S or T, the residue at position 615 is G, and the residue at position 616 is L. In other embodiments, the residue at position 614 is E, the residue at position 615 is G, and the residue at position 616 is L or A. In some embodiments, position 614 is S, position 615 is G, and position 616 is A. In some embodiments, the engineered polymerase may further comprise an A, G, or S at position 613 and/or position 617. In some embodiments, the engineered polymerase may further comprise a T at position 613 and/or position 617. In some embodiments, the residue at position 667 is Y. In some embodiments, an engineered polymerase as described in the present paragraph further comprises an N at position 655, M at position 657, K at position 681, N at position 742, and R at position 747.

In some embodiments, an engineered polymerase that that can incorporate a 3′-blocking nucleotide has at least 70%, at least 75%, at least 80%, or at least 85% identity to a naturally occurring family A polymerase. In some embodiments, the engineered polymerase has at least 90% identity to a naturally occurring family A polymerase. In further embodiments, the engineered polymerase has at least 95% identity to a naturally occurring family A polymerase. In some embodiments, the polymerase has no more than 99% identity to a naturally occurring polymerase, but at least 70%, at least 75%, at least 80%, at least 85% identity, or at least 90%, or 95% identity to a naturally occurring family A polymerase. In some embodiments, the engineered polymerase that incorporates a 3′-blocked nucleotide has at least 70%, at least 75%, at least 80%, or at least 85% identity; or at least 90% identity or 95% identity to a naturally occurring family A polymerase and comprises a substitution as described herein at a position corresponding to position 614, 615, and 616 of SEQ ID NO:1; and a substitution at a position corresponding to position 667 of SEQ ID NO:1.

In some embodiments, an engineered polymerase that can incorporate a 3′-blocking nucleotide comprises a polypeptide sequence having at least 70%, at least 75%, at least 80%, or at least 85% identity to SEQ ID NO:1. In some embodiments, the engineered polymerase comprises a polypeptide sequence having at least 90% identity to SEQ ID NO:1. In further embodiments, the engineered polymerase comprises an amino acid sequence having at least 95%, or greater, identity to SEQ ID NO:1.

In some embodiments, the polymerase comprises a polypeptide sequence having at least 95% identity to SEQ NO:2, wherein the polypeptide sequence comprises S at position 614, G at position 615, A at position 616, N at position 655, M at position 657, Y at position 667, K at position 681, N at position 742, and R at position 747.

(SEQ ID NO: 2)
ALEEAPWPPPEGAFVGFVLSRKEPMWADLLALAAARGGRVHRAPEPYKALRDLKEARGLLAK
DLSVLALREGLGLPPGDDPMLLAYLLDPSNTTPEGVARRYGGEWTEEAGERAALSERLFANL
WGRLEGEERLLWLYREVERPLSAVLAHMEATGVRLDVAYLRALSLEVAEEIARLEAEVFRLA
GHPFNLNSRDQLERVLFDELGLPAIGKTEKTGKRSTSAAVLEALREAHPIVEKILQYRELTK
LKSTYIDPLPDLIHPRTGRLHTRFNQTATATGRLSSSDPNLQNIPVRTPLGQRIRRAFIAEE
GWLLVALDYSQSGARVLAHLSGDENLIRVFQEGRDIHTETASWMFGVPREAVNPMMRRAAKT
INYGVLYGMSAHRLSQKLAIPYEEAQAFIERYFQSFPKVRAWIEKTLEEGRRRGYVETLFGR
RRYVPDLEARVKSVRNAAERRAFNMPVQGTAADLMKLAMVKLFPRLEEMGARMLLQVHDELV
LEAPKERAEAVARLAKEVMEGVYPLAVPLEVEVGIGEDWLSAKE

A polymerase may be produced by recombinant DNA technology using e any expression system, i.e., bacterial, archaeal, or eukaryotic, including yeast, mammalian, and the like. Illustrative expression systems are described, for example, in a number of manuals such as Sambrook & Russell, Molecular Cloning, A Laboratory Manual (4^{th} Ed, 2012); and Current Protocols in Molecular Biology (Ausubel, et al., John Wiley and Sons, New York, 1987-Volume 133, December 2020). Accordingly the disclosure additionally provided polynucleotides that encode a variant family A polymerase, e.g., a Taq polymerase, as described herein that incorporates 3′-blocked nucleotides; expression vectors that comprise such polynucleotides; and genetically modified host cells that express proteins encoded by the polynucleotides.

### Analysis of Variants

A variant as described herein can be analyzed for the ability to incorporate a 3′ blocking nucleotide, e.g., as described in the Examples section. In some embodiments, a variant is tested for the ability to incorporate a 3′-phosphate-blocked nucleotide. In some embodiments, the variant is analyzed for the ability to incorporate an alternative 3′-blocked nucleotide such as a 3′-O-nitrobenzyl-modified nucleotide. An illustrative assay to assess incorporation of a 3′-blocking nucleotide, including examples of reactions conditions, is detailed below. This particular assay involves two incorporation steps: the first is for the test 3′-blocked nucleotide and polymerase and the second is for the reporter nucleotide using a different polymerase. Primer and template oligonucleotides are combined at a final concentration of 2 μM each in Tris buffered saline, pH 7.5 at room temperature for a time sufficient to allow hybridization of the primer and template, e.g., an hour. The reaction components of the polymerase extension can be divided into two parts; one containing the enzyme and reaction buffer and the other with nucleotides, primer and template. The two reaction components are incubated at 55° C. in a PCR machine for 1 min before the nucleotides, primer and template are added to the enzyme mix, followed by immediate mixing. In this illustrative assay, the final reaction mix is 10 mM Tris buffer pH 7.5, 50 mM KCl, 5 mM MgSO₄, 1 μM MnCl₂, 0.1 μg/μ BSA, 0.05% triton X-100, and 3 μM nucleotide. The primer and template are at a final concentration of 0.1 μM each and the enzyme is typically at a concentration of 0.05 μg/l. After 20 seconds the reaction is stopped by the addition of EDTA to a final concentration of 20 mM. The reaction mix is then allowed to bind to streptavidin coated wells of a 96-well microplate for 1 hour after which time the wells are rinsed, e.g., several times with 5×SSC buffer, adjusted to pH 7.0, followed by several rinses in 50 mM NaCl, 50 mM Tris buffer pH 8.8.

The presence of incorporated blocking terminator is detected by the incorporation of a reporter nucleotide. Incorporation of labeled reporter nucleotides can be performed, e.g., with a 9° N DNA polymerase variant that accepts base-labeled, 2′,3′ dideoxy-modified nucleotides. Reporter nucleotides, e.g., at a concentration of 0.5 μM, can be modified with a fluorescent reporter such as Cy5 or Cy3 attached to the base and the incorporation reaction of reporter nucleotide performed at 55° C. for 5 min under reaction conditions that include 50 mM NaCl in 50 mM Tris buffer pH8.8. The plate wells are then rinsed with 5×SSC buffer pH 7 before multiple rinses in Tris buffered NaCl pH 8.8. The fluorescence intensity is scanned at the appropriate excitation and emission settings for the Cy3 and Cy5 dyes.

The reporter nucleotides are complementary to the P1 (first position after the primer terminus) and/or the P2 position (second position after the primer terminus) of the template. If the test nucleotide successfully incorporated at P1 then the P1 reporter is generally blocked from incorporating at P1. Comparison of the reporter signal to signal intensities from a well that does not contain a test nucleotide are used to determine the percentage of incompletion of incorporation and set as 100% incompletion of incorporation. The minimum intensity is established by no added polymerase to the reporter incorporation mix. Similarly, a 100% P2 intensity level is established from a no-test-nucleotide reaction mix and a primer extended by 1 base and the minimum P2 intensity level by no-added polymerase to the reporter incorporation mix. By reporting the P2 intensity concurrently, it is possible to estimate the level of un-blocked nucleotide incorporation since P2 intensity requires an unblocked P1 nucleotide to be incorporated.

A variant described herein typically exhibits at least 20%, at least 30%, at least 40%, at least 50%, at least 60%, at least 70%, at least 75%, at least 80%, at least 85%, or at least a 90% or 95%, or greater, efficiency in incorporating 3′-blocked terminator nucleotides under assay conditions described above. One of skill understand that incorporation of 3′-blocked terminator nucleotide can also be assessed using alternative assays. Other methods for determining incorporation efficiency include acrylamide gel size-separation analysis of primers extended in solution phase by either 1, or 2, or more positions. Mass spectrometry analysis of extended primer length also can be used to assess incorporation of nucleotides. Additional methods for determining incorporation efficiency can rely on fluorescent labels attached to the base, for which it has been established that the fluorescent label modification does not interfere with incorporation potential.

A variant polymerase “efficiently incorporates” a 3′-blocked nucleotide, e.g., a 3′-phosphate-blocked nucleotide, when it has at least a 20% efficiency, or at least 50% efficiency, or at least 70% efficiency, in incorporating 3′-blocked terminator nucleotides in one minute when assayed under the conditions described above. In some embodiments, e.g., for use in sequencing applications, a variant polymerase may have at least 90%, at least 95%, or at least 99% efficiency in incorporating 3′-blocked terminator nucleotides, e.g., 3′-phosphate-blocked nucleotides, in one minute when assayed under conditions described above. A variant polymerase typically may have at least 20% efficiency, or at least 50% efficiency, or at least 70% efficiency, in incorporating each of the 3′ blocked nucleotides (i.e., each of dATP, dTTP, dCTP, or dATP 3′-blocked nucleotides). A variant polymerase that incorporates 3′-blocked nucleotides, e.g., 3′-phosphate-blocked nucleotides, generally has a low preference for natural nucleotides vs the 3′-blocked nucleotides. In some embodiments, a variant polymerase of the invention has no preference for a natural nucleotide vs a counterpart 3′-blocked nucleotide, or has <2× or <3×, <4× or <5× preference for natural nucleotides when measure using the assay described above. In addition, a variant polymerase of the invention that incorporates 3′-blocked nucleotide typically exhibits specificity for complementary bases, e.g., is >5×, >10× or >30× to >100× more specific for complementary bases.

### Kits/Methods of Using Variant Polymerases

The disclosure additionally provide kits that include a variant polymerase as disclosed herein. In some embodiment, the kit further comprises a 3′-blocked oligonucleotide and optionally, one or more buffers for performing nucleotide polymerization. In some embodiments, the kit comprises reagents for sequencing.

In some embodiments, the disclosed polymerase compositions and kits comprising such compositions, can be used to obtain sequence information from a nucleic acid molecule, for example in massively parallel sequencing methods. In some embodiments, a variant polymerase as disclosed herein is employed in sequencing by synthesis in which a reversible 3′-terminator nucleotide is employed. In such a method successive nucleotides are incorporated into a chain. The presence of the 3′ blocked nucleotide prevents incorporation of the next nucleotide in the nucleic acid chain. Following identification of the 3′-blocked nucleotide, the blocking substituent may then be removed, e.g., by cleavage, to provide a free 3′ hydroxyl to allow for incorporation of the next nucleotide.

In some embodiments, the 3′ blocked nucleotide is a 3′-phosphate blocked nucleotide. In some instances, such a 3′ phosphate-blocked nucleotide is removed by enzymatic cleavage with a phosphatase, including, but not limited to, phosphatases such as Shrimp Alkaline Phosphatase, Antarctic phosphatase (New England Biolabs), Calf-intestinal phosphatase (Promega Corp.), or Fast AP (Thermo-Fisher). In some embodiments, removal of 3′ phosphate group can be achieved using T4 polynucleotide kinase (T4-PNK).

In some embodiments, the polymerase may be employed in single molecule sequencing.

In some embodiments, the polymerase may be employed for other purposes e.g., using a 3′-blocked nucleotide for labeling a nucleic acid.

In some embodiments, a variant polymerase of the present disclosure is used with a reversible 3′-blocked nucleotide analog. Such analogues include compounds represented by Formula I:

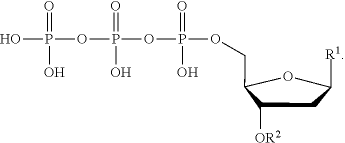

In Formula I, R¹ is a nitrogenous base suitable for use as a nucleoside base. For example, R¹ can be a base selected from the group consisting of adenine (A), cytosine (C), guanine (G), thymine (T), uracil (U), inosine (I), and derivatives of these. In some examples, R¹ can be selected from the group consisting of

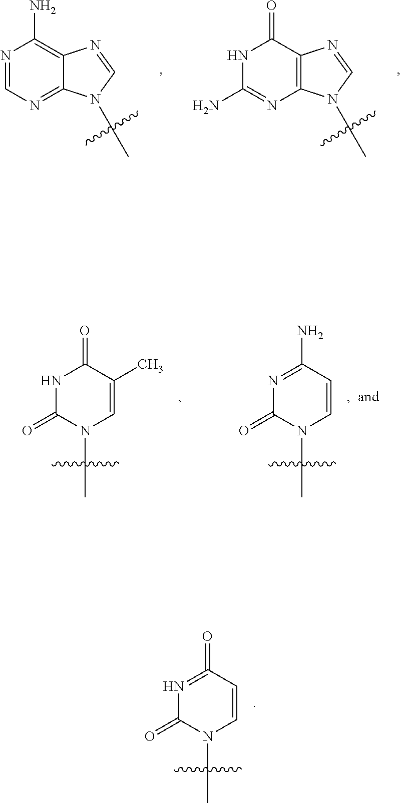

In Formula I, R² is a blocking group (also referred to herein as a 3′-blocking group). As described above, the term “blocking group” refers to any group that can be cleaved to provide a hydroxyl group at the 3′-position of the nucleotide analogue. The blocking group can be cleavable by physical means, chemical means, heat, and/or light. Optionally, the blocking group is cleavable by enzymatic means.

In some embodiments, R² can be a substituted or unsubstituted aryl or heteroaryl group. Optionally, R² can be a substituted or unsubstituted benzyl group, optionally having the following structure:

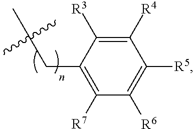

wherein:
n is 0, 1, 2, 3, or 4; and
R³, R⁴, R⁵, R⁶, and R⁷ are each independently selected from hydrogen, halogen, cyano, nitro, trifluoromethyl, alkoxy, aryloxy, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl.

Optionally, R² can be a nitrobenzyl blocking group. Optionally, R² can be selected from the following structures:

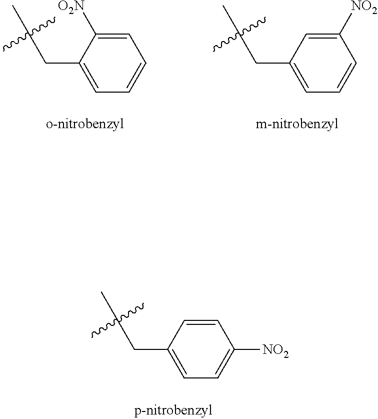 [SMILES: *Cc1ccccc1[N+](=O)[O-] ; *Cc1cccc([N+](=O)[O-])c1 ; *Cc1ccc([N+](=O)[O-])cc1]

In some embodiments, R² can be a phosphate-containing blocking group, a phosphonate-containing blocking group, or a phosphinate-containing blocking group. For example, the blocking group can be —PO₂H or —PO₃H₂. In some embodiments, the R² is an amino-containing blocking group (e.g., —NH₂). In some embodiments, R² is an allyl-containing blocking group (e.g., —CH₂CH═CH₂). In some embodiments, R² is an azido-containing blocking group (e.g., —CH₂N₃). In some embodiments, R² is an alkoxy-containing blocking group (e.g., —CH₂OCH₃). In some embodiments, R² is polyethylene glycol (PEG). The R² groups described above can be substituted or unsubstituted. In some embodiments, R² is a substituted or unsubstituted alkyl (i.e., a substituted or unsubstituted hydrocarbon).

Optionally, R² can be a blocking group selected from the group consisting of:

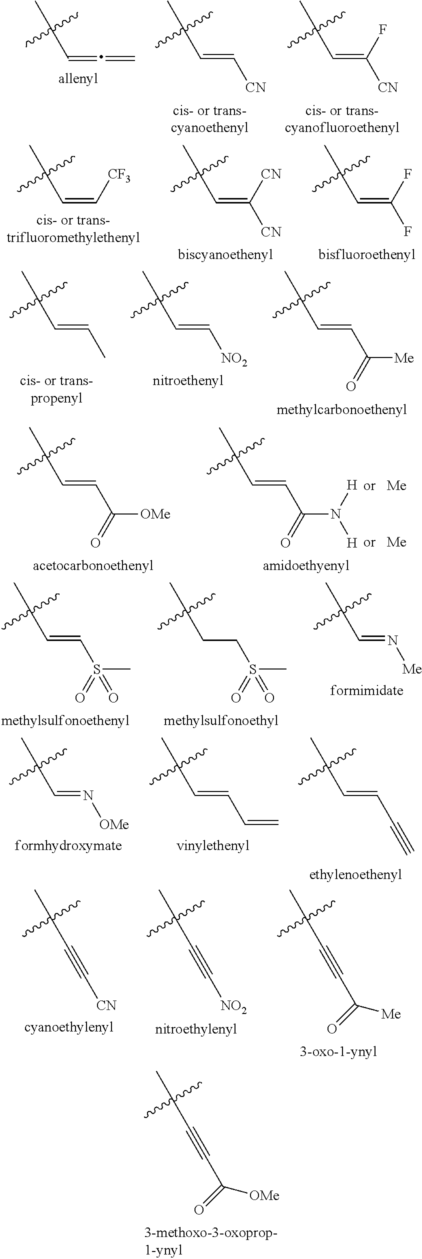

The compounds described herein can be prepared in a variety of ways known in the art of organic synthesis or variations thereon as appreciated by those skilled in the art. The compounds described herein can be prepared from readily available starting materials. Optimum reaction conditions may vary with the particular reactants or solvents used, but such conditions can be determined by one skilled in the art.

Variations on Formula I and the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection and the selection of appropriate protecting groups can be determined by one skilled in the art. The chemistry of protecting groups can be found, for example, in Wuts, Greene's Protective Groups in Organic Synthesis, 5th Ed., Wiley & Sons, 2014, which is incorporated herein by reference in its entirety. The synthesis and subsequent testing of various compounds as described herein to determine efficacy is contemplated.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

Optionally, the compounds described herein can be synthesized by using commercially available dNTPs. The 3′-position of the dNTPs can be blocked by reacting the compounds with a protecting group.

### Sequencing by Synthesis (SBS) Using Reversible Terminator Deoxyribonucleotides Comprising a Phosphate 3′ Blocking Group

Polymerases disclosed herein may be used in any suitable application in which deoxyribonucleotides (dNTPs) are combined to produce or extend a nucleic acid polymer. In some cases polymerases are used in massively parallel DNA sequencing (MPS) methods including sequencing-by-synthesis (SBS) methods. In some cases, the polymerase variants disclosed herein are uses in SBS methods in which a primer is extended by template-directed incorporation of reversible terminator nucleotides (RTs). In one aspect, the reversible terminator nucleotides include a 3′ phosphate blocking group. SBS requires multiple cycles of controlled (e.g., one at a time) incorporation of nucleotides based on base complementarity to the polynucleotide being sequenced. After addition of an RT to the terminus of the extended primer the incorporation may be detected. Following removal of the 3′-blocking group from the terminal nucleotide and regeneration of a 3′-OH terminus, the process can then be repeated in a new cycle. An SBS sequencing reaction may comprise 10-200 or more cycles of incorporation.

In a commonly used SBS approach, each RT comprises (1) a blocking group that ensures that only a single base can be added by a DNA polymerase enzyme to the 3′ end of a growing DNA strand (“GDS”, also called an “extended primer”), and (2) a fluorescent or luminescent label that can be detected by a camera. See, e.g. Bentley et al., Nature 456, 53-59, 2008. The fluorescent or luminescent label may be linked to the nucleotide base or sugar moiety by a cleavable linker. In this approach the RT is a “labeled RT.”

Another approach to SBS-based MPS uses directly- or indirectly-labeled affinity reagents (e.g., monoclonal antibodies or aptamers) that specifically recognize and identify the terminal base of an extended DNA primer in which a nonlabeled RT is incorporated. See Drmanac, et al. bioRxiv, doi:10.1101/2020.02.19.953307 (Feb. 20, 2020), Drmanac et al., U.S. Pat. Application Publication US20180223358, and International publication WO 2020/097607, each of which is incorporated herein by reference. A commercial embodiment of this approach is referred to as “CoolMPS®.” In this approach each RT comprises a modified nucleotide that includes a blocking group that ensures that only a single base can be added by a DNA polymerase enzyme to the 3′ end of a growing DNA copy strand but is not attached (e.g., via a linker) to a fluorescent or luminescent label. In this approach the RT is a “nonlabeled RT” or “NLRT.”

In one approach, it is contemplated that the labeled RT or non-labeled RT includes a 3′ blocking group that is a phosphate blocking group. In one approach it is contemplated that the labeled RT or non-labeled RT includes a 3′ blocking group that is a phosphate blocking group and the labeled RT or non-labeled RT is incorporated into the extended primer by a DNA polymerase variant described herein. In one approach it is contemplated that the labeled RT or non-labeled RT includes a 3′ blocking group that is a phosphate blocking group and is incorporated into the extended primer by a DNA polymerase other than a polymerase variant described herein. It is also contemplated that a DNA polymerase variant described herein is used to incorporate a labeled RT or non-labeled RT that has a 3′ blocking group other than phosphate (e.g., an azidomethyl- or a 3′-O-nitrobenzyl-modified nucleotide). In one approach it is contemplated that sequencing is carried out using a set of nucleotide analogs (e.g. a set of 4 nucleotide analogs comprising different bases) in which one or more nucleotide analogs comprises a 3′-phosphate blocking group and one or more nucleotide analogs comprises a blocking group that is not a 3′-phosphate blocking group.

### Labeled RTs

In one approach, sequencing is conducted using at least one labeled nucleotide analog that includes a 3′ phosphate blocking group. In this approach the labeled nucleotide analog is incorporated into the extended primer and the incorporation is detected based on a fluorescent or luminescent signal from the label. Following this, the label is removed, e.g., by cleavage of the linker connecting label to the nucleoside base or sugar, and the 3′-OH is unblocked, e.g., by cleavage of the 3′ blocking group, resulting in regeneration of the 3′ OH group. The cleaved 3′ blocking group and released label are washed away prior to the next cycle of incorporation.

### Non-Labeled RTs (NLRTs)

In one approach, sequencing is conducted using at least one NLRT that includes a 3′ phosphate blocking group. In this approach the NLRT is incorporated into the extended primer. The incorporation is detected using a directly- or indirectly-labeled affinity reagent (e.g., antibody or aptamer) that specifically binds to the incorporated NLRT based on affinity for a specified nucleobase, affinity for the phosphate blocking group, or affinity for a nucleotide characterized by both a specified nucleobase and a phosphate blocking group. Following this (1) the directly or indirectly labeled affinity reagent is removed and (2) the 3′-phosphate group is removed resulting in regeneration of the 3′ OH group. The affinity reagent and phosphate group are washed away. The affinity reagent and phosphate group may be removed in either order or simultaneously. See, e.g., U.S. Pat. Application Publication US 2018/0223358, and International publication WO 2020/097607.

In this approach, affinity reagents may be monoclonal antibodies (mAbs). Monoclonal antibodies that recognize nucleotide analogs with 3′ phosphate blocking groups may be prepared by art known means. See Example 5, below. Antibodies may be fluorescently labeled by an NHS ester reaction to lysine amino acids within the protein IgG heavy and light chains as previously described (Drmanac et al, bioRxiv, 2020, supra). In some embodiments, a polymerase as described herein is used for sequencing performed with 3′-phosphate-blocked nucleotide analogs in which the affinity agent is a monoclonal antibody that recognizes the blocked nucleotide.

### Removal of Phosphate Blocking Group

The enzymatic cleavage of DNA 3′ phosphate groups can be achieved by treatment with phosphatases including, but not limited to Shrimp Alkaline Phosphatase, Antarctic phosphatase (New England Biolabs), Calf-intestinal phosphatase (Promega Corp.), and Fast AP (Thermo-Fisher). These and other phosphatases are commercially available and conditions for enzymatic removal of phosphate are known. A kinase, such as T4 polynucleotide kinase (T4-PNK) can also be used to remove 3′ phosphate groups (Cameron et al., 1977, 3′-Phosphatase Activity in T4 Polynucleotide Kinase. Biochemistry. 16, 5120-5126). See Example 5 below.

### Removal of Affinity Reagents

Following a detection step, affinity reagents may be removed using any suitable method including, for example, methods described in Drmanac, et al. bioRxiv, doi:10.1101/2020.02.19.953307, posted Feb. 20, 2020; Drmanac et al., PCT publication WO 2020/097607, and U.S. Pat. Application Publication US20180223358.

Although the foregoing has been described in some detail by way of illustration and example, one of skill in the art will appreciate that certain changes and modifications may be practiced within the scope of the appended claims.

### EXAMPLES

### Example 1. Incorporation of F667Y Mutations is Class A Motif Mutations

This example describes engineered Taq polymerase to generate enzymes that can incorporate 3′ Phosphate. We included a mutation F667Y change with mutations I614E and E615G; and with mutations I614E, E615G, and L616A We demonstrated that inclusion of F667Y dramatically improved efficiency of 3′ phosphate incorporation. Inclusion of F667Y and L616A mutation in a polymerase background having mutations I614E, E615G, D655N, L657M, E681K, E742N, and M747R (SFSFr52 compared to SF73) provided an order of magnitude more efficiency than a polymerase having only I614E, E615G, and F667Y). Introducing L616A also provided substantial improvement of about 3-4 fold (SFr53 vs SFM19r5).

TABLE 1
Polymerase variants and associated amino acid changes. All variants are
derived from Taq DNA polymerase and SF designated variants are derived
from the Stoffel fragment (Lawyer, et al, PCR Methods Appl. 2, 275-287, 1993).
“Pos'n” in column 2 refers to the position in the Taq polymerase sequence;
Numeric column headers refer to position in Taq polymerase sequence)

| Name | Pos'n | 614 | 615 | 616 | 617 | 655 | 657 | 681 | 742 | 747 | 667 | 518 | 583 | 520 | 540 |
|---|---|---|---|---|---|---|---|---|---|---|---|---|---|---|---|
| SFm19 | I614E | E | G | L | R | D | L | E | E | M | F | V | N | E | K |
| | E615G | | | | | | | | | | | | | | |
| SFm19.2 | I614A | A | G | L | R | D | L | E | E | M | F | V | N | E | K |
| | E615G | | | | | | | | | | | | | | |
| SFm19.3 | I614E | E | G | A | R | D | L | E | E | M | F | V | N | E | K |
| | E615G | | | | | | | | | | | | | | |
| | L616A | | | | | | | | | | | | | | |
| SFm19.4 | I614D | D | G | L | R | D | L | E | E | M | F | V | N | E | K |
| | E615G | | | | | | | | | | | | | | |
| SFm19.5 | I614A | A | G | A | R | D | L | E | E | M | F | V | N | E | K |
| | E615G | | | | | | | | | | | | | | |
| | L616A | | | | | | | | | | | | | | |
| SFm19.6 | I614S | S | G | L | R | D | L | E | E | M | F | V | N | E | K |
| | E615G | | | | | | | | | | | | | | |
| SFm19.7 | I614S | S | G | A | R | D | L | E | E | M | F | V | N | E | K |
| | E615G | | | | | | | | | | | | | | |
| | L616A | | | | | | | | | | | | | | |
| SFm19.8.46 | I614E | E | G | L | R | N | M | K | N | R | F | V | N | E | K |
| (SFm4.6) | E615G | | | | | | | | | | | | | | |
| | D655N | | | | | | | | | | | | | | |
| | L657M | | | | | | | | | | | | | | |
| | E681K | | | | | | | | | | | | | | |
| | E742N | | | | | | | | | | | | | | |
| | M747R | | | | | | | | | | | | | | |
| SFm19.9.43 | I614E | E | G | L | R | N | L | K | Q | R | F | A | S | E | K |
| (SFm4.3) | E615G | | | | | | | | | | | | | | |
| | V518A | | | | | | | | | | | | | | |
| | N583S | | | | | | | | | | | | | | |
| | D655N | | | | | | | | | | | | | | |
| | E681K | | | | | | | | | | | | | | |
| | E742Q | | | | | | | | | | | | | | |
| | M747R | | | | | | | | | | | | | | |
| SFm19r3 | I614E | E | G | A | R | N | M | K | N | R | F | V | N | E | K |
| | E615G | | | | | | | | | | | | | | |
| | L616A | | | | | | | | | | | | | | |
| | D655N | | | | | | | | | | | | | | |
| | L657M | | | | | | | | | | | | | | |
| | E681K | | | | | | | | | | | | | | |
| | E742N | | | | | | | | | | | | | | |
| | M747R | | | | | | | | | | | | | | |
| SFm19r4 | I614D | D | G | L | R | N | M | K | N | R | F | V | N | E | K |
| | E615G | | | | | | | | | | | | | | |
| | D655N | | | | | | | | | | | | | | |
| | L657M | | | | | | | | | | | | | | |
| | E681K | | | | | | | | | | | | | | |
| | E742N | | | | | | | | | | | | | | |
| | M747R | | | | | | | | | | | | | | |
| SFm19r5 | I614E | E | G | L | R | N | M | K | N | R | Y | V | N | E | K |
| | E615G | | | | | | | | | | | | | | |
| | D655N | | | | | | | | | | | | | | |
| | L657M | | | | | | | | | | | | | | |
| | F667Y | | | | | | | | | | | | | | |
| | E681K | | | | | | | | | | | | | | |
| | E742N | | | | | | | | | | | | | | |
| | M747R | | | | | | | | | | | | | | |
| SF71 | I614E | E | G | L | R | D | L | E | E | M | Y | V | N | E | K |
| | E615G | | | | | | | | | | | | | | |
| | F667Y | | | | | | | | | | | | | | |
| SF72 | I614A | A | G | A | R | D | L | E | E | M | Y | V | N | E | K |
| | E615G | | | | | | | | | | | | | | |
| | L616A | | | | | | | | | | | | | | |
| | F667Y | | | | | | | | | | | | | | |
| SF73 | I614S | S | G | A | R | D | L | E | E | M | Y | V | N | E | K |
| | E615G | | | | | | | | | | | | | | |
| | L616A | | | | | | | | | | | | | | |
| | F667Y | | | | | | | | | | | | | | |
| SFr51 | I614S | S | G | L | R | N | M | K | N | R | Y | V | N | E | K |
| | E615G | | | | | | | | | | | | | | |
| | D655N/ | | | | | | | | | | | | | | |
| | L657M/ | | | | | | | | | | | | | | |
| | F667Y/ | | | | | | | | | | | | | | |
| | E681K/ | | | | | | | | | | | | | | |
| | E742N/ | | | | | | | | | | | | | | |
| | M747R | | | | | | | | | | | | | | |
| SFr52 | I614S | S | G | A | R | N | M | K | N | R | Y | V | N | E | K |
| | E615G | | | | | | | | | | | | | | |
| | L616A | | | | | | | | | | | | | | |
| | D655N | | | | | | | | | | | | | | |
| | L657M | | | | | | | | | | | | | | |
| | F667Y | | | | | | | | | | | | | | |
| | E681K | | | | | | | | | | | | | | |
| | E742N | | | | | | | | | | | | | | |
| | M747R | | | | | | | | | | | | | | |
| SFr53 | I614E | E | G | A | R | N | M | K | N | R | Y | V | N | E | K |
| | E615G | | | | | | | | | | | | | | |
| | L616A | | | | | | | | | | | | | | |
| | D655N | | | | | | | | | | | | | | |
| | L657M | | | | | | | | | | | | | | |
| | F667Y | | | | | | | | | | | | | | |
| | E681K | | | | | | | | | | | | | | |
| | E742N | | | | | | | | | | | | | | |
| | M747R | | | | | | | | | | | | | | |
| SFr54 | I614E | E | G | G | R | N | M | K | N | R | Y | V | N | E | K |
| | E615G | | | | | | | | | | | | | | |
| | L616G | | | | | | | | | | | | | | |
| | D655N | | | | | | | | | | | | | | |
| | L657M | | | | | | | | | | | | | | |
| | F667Y | | | | | | | | | | | | | | |
| | E681K | | | | | | | | | | | | | | |
| | E742N | | | | | | | | | | | | | | |
| | M747R | | | | | | | | | | | | | | |
| SFr55 | E615G | I | G | A | R | N | M | K | N | R | Y | V | N | E | K |
| | L616A | | | | | | | | | | | | | | |
| | D655N | | | | | | | | | | | | | | |
| | L657M | | | | | | | | | | | | | | |
| | F667Y | | | | | | | | | | | | | | |
| | E681K | | | | | | | | | | | | | | |
| | E742N | | | | | | | | | | | | | | |
| | M747R | | | | | | | | | | | | | | |
| SFr56 | I614E | E | G | A | F | N | M | K | N | R | Y | V | N | E | K |
| | E615G | | | | | | | | | | | | | | |
| | L616A | | | | | | | | | | | | | | |
| | R617F | | | | | | | | | | | | | | |
| | D655N | | | | | | | | | | | | | | |
| | L657M | | | | | | | | | | | | | | |
| | F667Y | | | | | | | | | | | | | | |
| | E681K | | | | | | | | | | | | | | |
| | E742N | | | | | | | | | | | | | | |
| | M747R | | | | | | | | | | | | | | |
| SFr57 | E615G | I | G | G | R | N | M | K | N | R | Y | V | N | E | K |
| | L616G | | | | | | | | | | | | | | |
| | D655N | | | | | | | | | | | | | | |
| | L657M | | | | | | | | | | | | | | |
| | F667Y | | | | | | | | | | | | | | |
| | E681K | | | | | | | | | | | | | | |
| | E742N | | | | | | | | | | | | | | |
| | M747R | | | | | | | | | | | | | | |
| SFr58 | I614E | E | G | A | K | N | M | K | N | R | Y | V | N | E | K |
| | E615G | | | | | | | | | | | | | | |
| | L616A | | | | | | | | | | | | | | |
| | R617K | | | | | | | | | | | | | | |
| | D655N | | | | | | | | | | | | | | |
| | L657M | | | | | | | | | | | | | | |
| | F667Y | | | | | | | | | | | | | | |
| | E681K | | | | | | | | | | | | | | |
| | E742N | | | | | | | | | | | | | | |
| | M747R | | | | | | | | | | | | | | |
| T60 | I614E | E | G | A | R | N | M | K | N | R | Y | V | N | E | K |
| | E615G | | | | | | | | | | | | | | |
| | L616A | | | | | | | | | | | | | | |
| | D655N | | | | | | | | | | | | | | |
| | L657M | | | | | | | | | | | | | | |
| | F667Y | | | | | | | | | | | | | | |
| | E681K | | | | | | | | | | | | | | |
| | E742N | | | | | | | | | | | | | | |
| | M747R | | | | | | | | | | | | | | |
| SFr80 | I614E | E | G | G | R | N | M | K | N | R | F | V | N | E | K |
| | E615G | | | | | | | | | | | | | | |
| | L616G | | | | | | | | | | | | | | |
| | D655N | | | | | | | | | | | | | | |
| | L657M | | | | | | | | | | | | | | |
| | E681K | | | | | | | | | | | | | | |
| | E742N | | | | | | | | | | | | | | |
| | M747R | | | | | | | | | | | | | | |
| SFr81 | E615G | I | G | G | R | N | M | K | N | R | F | V | N | E | K |
| | L616G | | | | | | | | | | | | | | |
| | D655N | | | | | | | | | | | | | | |
| | L657M | | | | | | | | | | | | | | |
| | E681K | | | | | | | | | | | | | | |
| | E742N | | | | | | | | | | | | | | |
| | M747R | | | | | | | | | | | | | | |
| SFr82 | I614G | G | G | G | R | N | M | K | N | R | F | V | N | E | K |
| | E615G | | | | | | | | | | | | | | |
| | L616G | | | | | | | | | | | | | | |
| | D655N | | | | | | | | | | | | | | |
| | L657M | | | | | | | | | | | | | | |
| | E681K | | | | | | | | | | | | | | |
| | E742N | | | | | | | | | | | | | | |
| | M747R | | | | | | | | | | | | | | |
| SFr83 | I614A | A | G | G | R | N | M | K | N | R | F | V | N | E | K |
| | E615G | | | | | | | | | | | | | | |
| | L616G | | | | | | | | | | | | | | |
| | D655N | | | | | | | | | | | | | | |
| | L657M | | | | | | | | | | | | | | |
| | E681K | | | | | | | | | | | | | | |
| | E742N | | | | | | | | | | | | | | |
| | M747R | | | | | | | | | | | | | | |
| SFr84 | I614E | E | G | G | K | N | M | K | N | R | F | V | N | E | K |
| | E615G | | | | | | | | | | | | | | |
| | L616G | | | | | | | | | | | | | | |
| | R617K | | | | | | | | | | | | | | |
| | D655N | | | | | | | | | | | | | | |
| | L657M | | | | | | | | | | | | | | |
| | E681K | | | | | | | | | | | | | | |
| | E742N | | | | | | | | | | | | | | |
| | M747R | | | | | | | | | | | | | | |
| Taq | | I | E | L | R | D | L | E | E | M | F | V | N | E | K |
| Thermos | del | I | E | L | R | D | L | E | E | M | Y | V | N | E | K |
| eq | 271:F | | | | | | | | | | | | | | |
| | 667Y | | | | | | | | | | | | | | |
| Taq 475 | E520G, | I | E | A | R | D | L | E | E | M | F | V | N | G | I |
| | K540I, | | | | | | | | | | | | | | |
| | L616A | | | | | | | | | | | | | | |

### General Plate Assay Design

Testing the incorporation efficiency of polymerases. Primer and template oligonucleotides were annealed in a 96 well streptavidin coated microplate. The template was 5′ biotinylated and bound to the streptavidin coated surface for 30-60 min in Tris-buffered saline at 0.1 uM template concentration. The primer was then hybridized to the bound template for 10 min at 1 uM primer concentration. Specific test nucleotide tri-phosphates with each one of the bases A, C, G or T were then incorporated to the template primers which were either complementary or mis-matched to the first position after the primer terminus (described as P1). After incorporation, the reaction mix was removed and the wells washed before addition of a second incorporation mix of labeled reporter nucleotides and polymerase. The reporter nucleotides were generally a dideoxy nucleotide with a fluorescent reporter such as Cy5 or Cy3 attached to the base. The reporter nucleotides were complementary to the P1 and/or P2 position (second position after the primer terminus) of the template. If the test nucleotide successfully incorporated at P1 then the P1 reporter is generally blocked from incorporating at P1. Comparison of the reporter signal to signal intensities from a well that did not contain a test nucleotide was used to determine the percentage of incompletion of incorporation. 100% intensity suggests no test nucleotide incorporation since maximum reporter can incorporate and 0% intensity suggests full occupancy of the P1 site by the test nucleotide. By reporting the P2 intensity concurrently, it is possible to estimate the level of un-blocked nucleotide incorporation since P2 intensity requires an unblocked P1 nucleotide to be incorporated.

### Incorporation of 3′ Phosphate Terminated Nucleotides.

Table 2(a) below shows the percent incorporation of a fluorescently labeled nucleotide at the first extended position from a primer terminus. The inclusion of no nucleotide in the test incorporation reaction is set at 100% since maximum incorporation of the reporter is possible. When 0.06 uM or 0.12 uM dNTP is included in the test incubation, some suppression of reporter intensity is seen under some conditions suggesting the natural nucleotide is partially occupying some available P1 positions. For BG9 polymerase at 0.12 uM natural dTTP nucleotide, a significant amount of occupancy is occurring (>95%) but with some of the variants less than 5% of sites are occupied by the natural nucleotide. When 3′ phosphorylated terminator is used the percent of available sites drops to less 5% for variants sfr51, sfr53 and sf73. Other variants also show falls of varying degrees. This suggests the 3′ phosphorylated nucleotide can be efficiently incorporated under these conditions. When the natural dNTP nucleotide is included with the 3′ phosphorylated nucleotide but at concentration of just 2% or 4% of the 3′ phosphorylated nucleotide little change is seen in the P1 reporter intensities for most of the variants. BG9 does not show significant incorporation of the 3′ phosphorylated nucleotide but still shows preference for the natural nucleotide. Even with the addition of natural nucleotide included with the 3′ phosphorylated nucleotide, the variants showed a preference for the modified nucleotide.

TABLE 2(a)
Incorporation efficiencies of 3′ phosphorylated nucleotides (in 1 mM
Mn⁺²). Values represent the percentage of incorporation of fluorescently
labeled reporter nucleotides relative to a maximum incorporation as
determined by no test nucleotide inclusion. Reporter nucleotides can
incorporate at P1 when the site is un-occupied by the test nucleotide.

| | P1 (T) | | | | | | |
|---|---|---|---|---|---|---|---|
| | sfr5 | sfr53 | sfr51 | sf71 | sf72 | sf73 | BG9 |
| 0 | 100.0 | 100.0 | 100.0 | 100.0 | 100.0 | 100.0 | 100.0 |
| 0.06 uM dTTP | 98.1 | 101.1 | 102.1 | 79.2 | 84.1 | 56.5 | 12.7 |
| 0.12 uM dTTP | 97.0 | 92.0 | 80.3 | 69.2 | 66.7 | 23.1 | 3.2 |
| 3P-dTTP | 5.7 | 2.9 | 2.8 | 25.6 | 6.7 | 3.4 | 93.5 |
| 3P dTTP + 0.06 uM dTTP | 7.6 | 2.5 | 2.2 | 22.2 | 5.5 | 2.9 | 18.2 |
| 3P dTTP + 0.12 uM dTTP | 6.9 | 2.9 | 2.5 | 13.2 | 4.8 | 2.5 | 3.6 |
| | P1 (G) | | | | | | |
| | sfr5 | sfr53 | sfr51 | sf71 | sf72 | sf73 | BG9 |
| 0 | 100.0 | 100.0 | 100.0 | 100.0 | 100.0 | 100.0 | 100.0 |
| 0.06 uM dGTP | 96.9 | 88.0 | 98.8 | 59.5 | 83.9 | 63.8 | 37.8 |
| 0.12 uM dGTP | 94.5 | 72.6 | 88.0 | 104.5 | 100.2 | 93.4 | 5.3 |
| 3P-dGTP | 3.1 | 3.5 | 2.7 | 2.7 | 3.1 | 2.5 | 92.2 |
| 3P dGTP + 0.06 uM dGTP | 5.4 | 3.5 | 2.2 | 3.4 | 1.6 | 2.3 | 41.1 |
| 3P dGTP + 0.12 uM dGTP | 6.7 | 2.6 | 3.8 | 3.1 | 2.8 | 2.9 | 6.2 |
| | P1 (A) | | | | | | |
| | sfr5 | sfr53 | sfr51 | sf71 | sf72 | | BG9 |
| 0 | 87.1 | 100.0 | 112.8 | 100.8 | 96.5 | | 96.1 |
| 0.06 uM dATP | 86.7 | 96.0 | 71.3 | 57.0 | 65.1 | | 61.6 |
| 0.12 uM dATP | 88.6 | 73.5 | 21.1 | 13.7 | 29.1 | | 26.0 |
| 3P-dATP | 2.8 | 3.7 | 2.4 | 5.4 | 3.4 | | 93.1 |
| 3P dATP + 0.06 uM dATP | 3.1 | 3.1 | 2.8 | 3.4 | 2.8 | | 43.5 |
| 3P dATP + 0.12 uM dATP | 3.0 | 3.1 | 3.3 | 3.3 | 2.6 | | 15.6 |

Table 2(b) shows the relative reporter intensities at the second position after the incorporation of the test nucleotide position. Incorporation of un-blocked nucleotide at P1 will show as increased reporter intensity at P2. This can be seen with the incorporation of natural nucleotide with BG9 polymerase and with some of the variants such as sf73 with 91.8% of maximum P2 intensity when 0.12 uM natural dTTP nucleotide was used. It is of note that some variants such as sfr53 with 1.9% P2 reporter intensity show very minimal P2 intensity suggesting low incorporation of natural dTTP at P1 even without a competing 3′ phosphorylated dTTP nucleotide present.

TABLE 2(b)
Reporter incorporation efficiencies at P2 with incorporation of 3′ phosphorylated
nucleotides at P1. Values represent the percentage of incorporation of fluorescently labeled
reporter nucleotides relative to a maximum incorporation as determined by no test nucleotide
inclusion and a primer that allows incorporation at P2. Reporter nucleotides can incorporate
at P2 when the site is un-occupied and the nucleotide incorporated at P1 is un-blocked.

| | P2 (P1 T) | | | | | | |
|---|---|---|---|---|---|---|---|
| | sfr5 | sfr53 | sfr51 | sf71 | sf72 | sf73 | BG9 |
| 0 | 1.8 | 1.4 | 1.4 | 1.6 | 1.5 | 1.9 | 1.6 |
| 0.06 uM dTTP | 2.0 | 1.7 | 2.6 | 21.7 | 22.3 | 48.1 | 114.0 |
| 0.12 uM dTTP | 2.8 | 1.9 | 10.2 | 50.7 | 50.3 | 91.8 | 122.6 |
| 3P-dTTP | 2.5 | 0.6 | 1.1 | 1.0 | 1.3 | 1.1 | 1.5 |
| 3P dTTP + 0.06 uM dTTP | 2.6 | 0.3 | 3.7 | 26.5 | 21.2 | 32.7 | 104.3 |
| 3P dTTP + 0.12 uM dTTP | 2.3 | 0.6 | 6.9 | 54.1 | 38.0 | 57.7 | 133.4 |
| | P2 (P1 G) | | | | | | |
| | sfr5 | sfr53 | sfr51 | sf71 | sf72 | sf73 | BG9 |
| 0 | 1.5 | 0.9 | 1.1 | 0.8 | 0.8 | 0.9 | 0.6 |
| 0.06 uM dGTP | 2.3 | 4.1 | 1.4 | 54.2 | 16.0 | 42.8 | 59.9 |
| 0.12 uM dGTP | 1.4 | 29.6 | 4.4 | 2.2 | 0.8 | 1.2 | 94.1 |
| 3P-dGTP | 9.0 | 1.5 | 0.5 | 2.0 | 2.1 | 1.8 | 3.4 |
| 3P dGTP + 0.06 uM dGTP | 11.1 | 8.1 | 1.1 | 9.7 | 6.1 | 10.2 | 59.0 |
| 3P dGTP + 0.12 uM dGTP | 10.2 | 20.9 | 2.5 | 20.5 | 9.0 | 19.3 | 101.5 |
| | P2 (P1 A) | | | | | | |
| | sfr5 | sfr53 | sfr51 | sf71 | sf72 | sf71 | BG9 |
| 0 | 0.2 | 0.3 | 0.1 | 0.1 | 0.1 | | −0.1 |
| 0.06 uM dATP | 0.3 | 10.4 | 35.0 | 53.9 | 41.1 | | 35.3 |
| 0.12 uM dATP | 0.4 | 26.5 | 84.6 | 89.1 | 67.3 | | 69.5 |
| 3P-dATP | 3.4 | 0.5 | 0.5 | 0.8 | 0.8 | 0.9 | 0.3 |
| 3P dATP + 0.06 uM dATP | 4.1 | 10.9 | 7.2 | 39.1 | 21.2 | 26.5 | 51.4 |
| 3P dATP + 0.12 uM dATP | 6.1 | 11.8 | 14.5 | 64.9 | 33.6 | 48.4 | 82.4 |

Table 3 shows incorporation efficiencies of nucleotides with the SFr51 variant with different times of incubation of the nucleotide reaction mix. A general decrease in percent incomplete with increasing time suggests a time dependence of the incorporation but certain aspects of the assay design may be limiting and preventing 100% incorporation (represented as 0% reporter intensity in Table 3).

TABLE 3
Incorporation efficiencies of nucleotides with the SFr51 variant.
Primer and 5′ biotinylated template were hybridized in solution
before the addition of a mix of enzyme and nucleotide in reaction
buffer. 3 uM nucleotide was reacted with 0.05 ug/ul enzyme for
the indicated times before the addition of EDTA to a final concentration
of 10 mM. The biotinylated template, with extended primer, was
then allowed to bind to streptavidin coated wells of a microplate.
After washing to remove excess unbound reaction components the
presence of incorporated blocking terminator was detected by
the incorporation of a reporter nucleotide.

| | 0.5 min | 1 min | 2 min | 3 min |
|---|---|---|---|---|
| 3′ phoshate-dATP | 5.6 | 5.8 | 5.6 | 4.3 |
| 2′ phoshate-dATP | 5.8 | 4.8 | 4.6 | 3.9 |
| dideoxy ATP | 13.6 | 11.4 | 9.6 | 6.0 |

Table 4 shows the incorporation efficiencies of multiple SF variants with Firebird 3′-ONH2 modified dGTP. All variants appeared to show significant incorporation. All of the variants had the F667Y mutation and several had the L616A mutation which were previously shown to support incorporation of nucleotides with the —ONH2 moiety (15).

TABLE 4
Incorporation of Firebird nucleotides with SF variants. Biotinylated template was
bound to streptavidin coated wells of a microplate. A primer was hybridized and then
extended with 3 uM firebird nucleotides for 2 minutes. 100% value at P1 represents no
incorporation of the test nucleotide as determined from values obtained by incubation of the
polymerase without nucleotides and 0% represents blocking of the P1 position by
incorporation of the firebird nucleotide. Similarly, a 100% value at P2 represents
incorporation of un-blocked nucleotide at P1.

| | | sfm19r5 | SF71 | SF72 | SF73 | SFr51 | SFr52 | SFr53 |
|---|---|---|---|---|---|---|---|---|
| 3′ONH2-dGTP | P1 | 2.2% | 2.0% | 2.0% | 0.9% | −0.7% | 1.3% | 1.0% |
| | P2 | 0.0% | 0.1% | 0.0% | 0.0% | 0.0% | −0.1% | 0.1% |

Table 5 shows the incorporation efficiencies of multiple SF variants with a 5 minute incubation time and with 5 uM nucleotide concentration. SFm19.7 was one of the better performing variants with only changes at the 614-616 steric gate region. Most notably the L616A modification. Modifications to the primer binding site amino acids also appeared to affect the incorporation efficiencies with SFm19r3 and SFm19r5 producing noticeably improved incorporation efficiencies. SFm19r5, the only variant with an additional F667Y amino acid change, appeared as the most effective for 3′ phosphate incorporation of this comparison set. It is of interest to note that in all cases (except SFm19.3) the P2 percentage incorporations were low suggesting the level of contaminating natural nucleotide is low and/or that the 3′ phosphate is stable to un-blocking under these test conditions. Here, low P2 incorporation percentage is indicative of the inability to add the P2 reporter because of a blocked nucleotide at P1. After cleavage, there is an increase of P2 intensity as would be expected if the 3′ phosphate blocking group was converted back to a 3′-OH to allow continued extension. The higher than expected P2 intensity with SFm19.3 could indicate some residual natural nucleotide in the enzyme preparation or an enhanced preference to incorporate the natural nucleotide.

TABLE 5
Incorporation of 3′-phosphate dTTP nucleotide with SF variants.
Biotinylated template was bound to streptavidin coated wells
of a microplate. A primer was hybridized and then extended with
5 uM nucleotides for 5 minutes at a set temperature of 60°
C. and with 0.05 ug/ul of enzyme. 100% value at P1 represents
no incorporation of the test nucleotide as determined from values
obtained by incubation of the polymerase without nucleotides
and 0% represents blocking of the P1 position by incorporation
of the test nucleotide. P2 Values represent the percentage of
incorporation of fluorescently labeled reporter nucleotides relative
to a maximum incorporation as determined by no test nucleotide
inclusion and a primer that allows incorporation at P2.

| Polymerase | P1 | P2 | P2 after cleavage | |
|---|---|---|---|---|
| SFm19.2 | 99.5% | 1.6% | ND | |
| | 99.6% | 1.7% | ND | |
| SFm19.3 | 78.9% | 12.6% | 15.4% | |
| | 90.6% | 9.8% | ND | |
| SFm19.4 | 86.8% | 1.1% | ND | |
| | 82.8% | 0.8% | ND | |
| SFm19.5 | 96.1% | 1.6% | ND | |
| | 92.4% | 1.3% | ND | |
| SFm19.6 | 72.2% | 1.5% | 33.5% | |
| | 74.5% | 1.4% | 35.1% | |
| SFm19.7 | 35.4% | 0.1% | 75.0% | |
| | 34.3% | 0.0% | 77.0% | |
| SFm19.8.46 | 69.2% | 0.9% | 28.2% | |
| | 68.4% | 0.8% | 26.1% | |
| SFm19.9.43 | 79.2% | 1.2% | 25.8% | |
| | 75.1% | 0.5% | 24.6% | |
| SFm19r3 | 18.0% | 0.1% | 85.2% | |
| | 21.3% | 0.2% | 83.3% | |
| SFm19r4 | 60.0% | 1.7% | 57.6% | |
| | 63.8% | 1.5% | 45.4% | |
| SFm19r5 | 2.7% | −0.1% | 107.0% | N1 |
| | 0.8% | −0.1% | 107.7% | N2 |
| | 0.6% | −0.1% | 105.1% | N3 |

Multiple SF variants were tested for incorporation efficiencies within a 2 minute reaction time as shown in Table 6. Variants SFr51, SFr52 and SFr53 showed the most complete incorporation with around 1% of available primer sites un-extended. These variants were modified to serine at position 614 and or alanine at 616 compared with SFm19r5. As with SFm19r5 they also possessed the F667Y mutation and mutations reported for improved 2′ modified primer acceptance (SFm 4.6 mutations). SF71, SF72 and SF73 did not possess these SFm 4.6 changes and appear to show slower incorporation efficiencies within the 2 minute incubation. SFm19 appears to not show incorporation of the 3′ phosphate under these conditions.

TABLE 6
Incorporation of 3′-phosphate dATP nucleotide with SF variants.
Biotinylated template was bound to streptavidin coated wells of
a microplate. A primer was hybridized and then extended with 3 uM
nucleotides for 2 minutes at a set temperature of 60° C. and
with 0.1 ug/ul of enzyme. 100% value at P1 represents no incorporation
of the test nucleotide as determined from values obtained by incubation
of the polymerase without nucleotides and 0% represents blocking
of the P1 position by incorporation of the test nucleotide.

| Polymerase | P1 |
|---|---|
| SFm19r5 | 22.0% |
| SF71 | 82.6% |
| SF72 | 62.7% |
| SF73 | 11.7% |
| SFr51 | 0.7% |
| SFr52 | 0.9% |
| SFr53 | 1.1% |
| SFm19 | 103.7% |

Multiple SF variants were further tested for incorporation efficiencies within a 20 second reaction time as shown in Table 7. Variants SFr51 and SFr52 showed the most complete incorporation with around 4% of available primer sites un-extended. These two mutants were modified to serine at position 614 and as with SFr5 they also possessed the F667Y mutation and mutations reported for improved 2′ modified primer acceptance (SFm 4.6). SF71, SF72 and SF73 did not possess these SFm 4.6 changes and appear to show slower incorporation efficiencies with SFr72 appearing not to incorporate detectible 3′ Phosphate under these conditions.

TABLE 7
Incorporation efficiencies of 3′ phosphate blocked dATP nucleotides
with multiple SF variants. Primer and 5′ biotinylated template
were hybridized in solution before the addition of a mix of enzyme
and nucleotide in reaction buffer. 3 uM nucleotide was reacted
with 0.05 ug/ul enzyme for 20 seconds at 55° C. before the
addition of EDTA to a final concentration of 10 mM. The biotinylated
template with extended primer was then allowed to bind to streptavidin
coated wells of a microplate. After washing to remove excess unbound
reaction components the presence of incorporated blocking terminator
was detected by the incorporation of labeled reporter nucleotides.
100% value at P1 represents no incorporation of the test nucleotide
as determined from values obtained by incubation without nucleotides
or polymerase and 0% represents blocking of the P1 position by
incorporation of the test nucleotide. Similarly, a 100% value
at P2 represents incorporation of un-blocked nucleotide at P1
and a 0% value represents blocked P1 incorporation or no incorporation.
P2 100% value was determined with a primer that allows P2 position
determination.

| Polymerase | P1% of max | P2% of max |
|---|---|---|
| SF71 | 77.3 | 0.4 |
| SF72 | 119.5 | 0.0 |
| SF73 | 71.1 | 0.6 |
| SFr51 | 4.3 | 0.3 |
| SFr52 | 4.2 | 0.3 |
| SFr53 | 8.2 | 1.8 |
| SFm19r5 | 27.7 | 2.7 |

### Amino Acid Changes at Residue 614, 615, 616, and 667 of Variants of Interest

Table 8 shows differences in amino acids at key positions of variants SFm51, SFm52 and SFm53. SFm 4-6 was previously reported with properties that promote incorporation of 2′ O-Me modified nucleotides (Chen et al., 2016, supra). Positions 616L and 667F are wild-type in this variant but positions 614 and 615 carry the SFm19 variations. Relative to SFm4-6 the SFm19r5 variant has one amino acid change at position 667. Conversion of position 667 to tyrosine (Y) from phenylalanine (F) position was previously reported as beneficial to the enhanced incorporation of 2′, 3′ dideoxy modified nucleotides.

TABLE 8
Amino acids at key positions of preferred variants and reference
polymerases. A; Alanine, E; Glutamate, I; Isoleucine, F; Phenylalanine,
G; Glycine, L, Leucine, S; Serine, Y, Tyrosine.

Taq sequence position

| Name | 614 | 615 | 616 | 667 |
|---|---|---|---|---|
| Taq | I | E | L | F |
| SFm19.8.46 (SFm 4.6) | E | G | L | F |
| SFm19r5 | E | G | L | Y |
| SFr51 | S | G | L | Y |
| SFr52 | S | G | A | Y |
| SFr53 | E | G | A | Y |

Table 9 shows that introduction of the F667Y modification to SFm4-6 resulted in an increase in 3′ phosphate modified nucleotide incorporation as evidenced by the decrease of incomplete sites from 74.8% to 11.5% within the 20 sec incorporation period under 10 uM manganese conditions. A manganese concentration of 1 uM showed a similar improvement with 91.1% incompletion reduced to 7.2% incompletion when including the F667Y modification. While maintaining the F667Y amino acid change, further modification in the motif-A region at position 614 from Glutamate to Serine in SFr51 resulted in improved incorporation efficiency under both manganese concentrations of around 1.8 to 5% incompletion. This was comparable to the single additional change at 616 in SFr53 of 4-5.5% incompletion or the double modification of 614 and 616 in SFr52 with incompletion values of 3-4.9%

TABLE 9
Incorporation efficiencies of 3′-phosphate blocked dATP nucleotides in preferred SF
variants. Primer and 5′ biotinylated template were hybridized in solution before the addition
of a mix of enzyme and nucleotide in reaction buffer. 3 uM nucleotide was reacted with 0.05
ug/ul enzyme for 20 seconds at 55° C. before the addition of EDTA to stop the reaction. The
biotinylated template with extended primer was then allowed to bind to streptavidin coated
wells of a microplate. After washing to remove excess unbound reaction components the
presence of incorporated blocking terminator was detected by the incorporation of labeled
reporter nucleotides. 100% value represents no incorporation of the test nucleotide as
determined from values obtained by incubation without nucleotides or polymerase and 0%
represents blocking of the P1 position by incorporation of the test nucleotide.

| | 10 uM | 1 uM | | | | | | | | | |
|---|---|---|---|---|---|---|---|---|---|---|---|
| | Mn++ | Mn++ | 614 | 615 | 616 | 655 | 657 | 681 | 742 | 747 | 667 |
| SFm19.8.46 | 74.80% | 91.10% | E | G | L | N | M | K | N | R | F |
| (SFm 4-6) | | | | | | | | | | | |
| SFm19r5 | 11.50% | 7.20% | E | G | L | N | M | K | N | R | Y |
| SFr51 | 1.80% | 5.00% | S | G | L | N | M | K | N | R | Y |
| SFr52 | 3.00% | 4.90% | S | G | A | N | M | K | N | R | Y |
| SFr53 | 5.50% | 4.00% | E | G | A | N | M | K | N | R | Y |

Incorporation Efficiencies of 3′ Phosphate Modified Nucleotides Across all 4 Base Types with 20 Second Incubation.

Table 10 shows the relative incorporation efficiencies of 7 variant enzymes across all 4 bases. The lower the percentage value the more complete the incorporation of the 3′ phosphate modified nucleotide in the 20 second incubation time. SFr51 with one change in the motif A region at 614S and SFr52 with two changes in the motif A region of 614S and 616A both showed generally improved levels of incorporation compared with the single 616A change in SFr53. All three variants (SFr51, SFr52 and SFr53) include the primer binding amino acid changes previously reported for SFm4-6 at positions 655, 657, 681, 742 and 747 (Chen et al., 2016, supra). In variants that do not include the primer binding amino acid changes described by Chen et al., SF71, SF72 and SF73, the SF73 variant that includes the 614S amino acid generally performed better than those with 614E or 614A.

Table 2(a) with 2 minute incorporations times and slightly different incorporation conditions (surface bound vs solution phase incorporation and 1 mM vs 1 μM Mn⁺²) also showed a general improvement of SFr51 compared with SFr53 for incorporation of the 3′ phosphorylated nucleotide. Table 2(a) also shows that SF73 with the 614S modification performed best for incorporation of 3′ phosphorylated dTTP and dGTP nucleotide incorporation.

Table 2(b) also demonstrates that SFr51 incorporated natural nucleotide to a lesser extent than SFr53 when spiked into the 3′ phosphorylated nucleotide at a 2% ratio for dGTP and dATP but not for dTTP or a 4% dATP spike. However, overall the SFr51 and SFr53 variants incorporated less natural nucleotide compared with the SF71, SF72 and SF73 variants when competition between 3′ phosphorylated nucleotide and natural nucleotide exists.

TABLE 10
Percentage incorporation efficiencies of 3′-phosphate blocked nucleotides in SF
variants. Primer and 5′ biotinylated template were hybridized in solution before the addition
of a mix of enzyme and nucleotide in reaction buffer. 3 uM nucleotide was reacted with 0.05
ug/ul enzyme for 20 seconds at 55° C. in a buffer containing 1 uM Mn⁺² before the addition of
EDTA to stop the reaction. The biotinylated template with extended primer was then allowed
to bind to streptavidin coated wells of a microplate. After washing to remove excess unbound
reaction components the presence of incorporated blocking terminator was detected by the
incorporation of labeled reporter nucleotides. 100% value represents no incorporation of the
test nucleotide as determined from values obtained by incubation without nucleotides or
polymerase and 0% represents blocking of the P1 position by incorporation of the test
nucleotide. Amino acids at key positions are shown for reference (shaded boxes).

| | A | C | G | T | 614 | 615 | 616 | 655 | 657 | 681 | 742 | 747 | 667 |
|---|---|---|---|---|---|---|---|---|---|---|---|---|---|
| SF71 | 77.3 | 66.8 | 68.8 | 94.7 | E | G | L | D | L | E | E | M | Y |
| SF72 | 119.5 | 59.0 | 21.5 | 84.3 | A | G | A | D | L | E | E | M | Y |
| SF73 | 71.1 | 20.5 | 4.3 | 17.9 | S | G | A | D | L | E | E | M | Y |
| SFr51 | 4.3 | 11.3 | 8.4 | 5.9 | S | G | L | N | M | K | N | R | Y |
| 2SFr52 | 4.2 | 9.2 | 9.5 | 3.8 | S | G | A | N | M | K | N | R | Y |
| SFr53 | 8.2 | 8.9 | 9.9 | 6.4 | E | G | A | N | M | K | N | R | Y |
| SFm19r5 | 27.7 | 11.2 | 11.4 | 10.2 | E | G | L | N | M | K | N | R | Y |
| SFm19.8.46 | | | | | E | G | L | N | M | K | N | R | F |
| (SFm 4-6) | | | | | | | | | | | | | |

### Example 2. SF Variant Incorporation Efficiencies of 3′-O-Nitrobenzyl Blocked Nucleotides

Table 11 shows the P1 incorporation efficiencies of 3′-O-nitrobenzyl (NB) modified nucleotides by several SF variant polymerases. In this assay the 0% incorporation value was set by the incorporation of 3′-O-nitrobenzyl modified nucleotides by a 9° N variant DNA polymerase (BG9) known to incorporate the modified nucleotide. The preferred variants SFr51 and SFr52 were both able to efficiently incorporate the NB-dCTP nucleotide with 1% residual un-incorporated in the 2 min incubation time period. NB-dATP and NB-dTTP were less efficient with between 21% to 43% residual un-incorporated targets. SFm19r5 and SFr53, both variants without the 614S modification, showed generally poor incorporation of 3′-O-nitrobenzyl within the 2 minute incubation period.

TABLE 11
Incorporation efficiencies of 3′ O-nitrobenzyl (NB) blocked
dATP, dCTP and dTTP nucleotides in preferred SF variants. Biotinylated
template was bound to streptavidin coated wells of a 96-well
microplate. A primer was then hybridized and extended with 2 uM
3′-nitrobenzyl modified nucleotides for 2 minutes at 55° C.
and in the presence of 100 uM MnCL₂. 100% value represents
no incorporation of the test nucleotide as determined from values
obtained by incubation of a 9°N variant polymerase (BG9) without
nucleotides for the same time period and a 0% value represents
blocking of the first position by incorporation of the 3′-
nitrobenzyl nucleotide by the 9°N variant polymerase.

| | NB_A | NB_C | NB_T |
|---|---|---|---|
| SFm19r5 | 93% | 60% | 102% |
| SFr51 | 21% | 1% | 43% |
| SFr52 | 29% | 1% | 23% |
| SFr53 | 78% | 67% | 83% |

This result demonstrate an importance of 614S amino acid independent of 3′ phosphate group (highly negatively charged group) allowing incorporation of the much bigger and chemically very different NB group (big benzyl ring, more hydrophobic). 614S provides more efficient incorporation of 3′ phosphate than 614E or 614A (comparing SFm19r5 with 614E, SF71 with 614A and SF72 and SFr51 both with 614S) further indicating that 614S is important not just to provide more space (smaller than E) but also that is chemically important (similar size to A but more effective than A). S has an —OH group that may be important for interactions with Mg++ when 3′-OH is replaced by a 3′ blocking group. Other similar amino acids may be used.

To create a larger polymerase pocket for most efficient incorporation of larger 3′ blocking groups (e.g., NB is overall more difficult to incorporate than the smaller phosphate or NH2 blocking groups, even by SFr52, which has 614S and 616A) an S (or a similar amino acid such as threonine) may be placed at positions 613 or 616 or 617 and replace 614S and/or 616A with 614G and/or 616G for maximal pocket opening and minimal other chemical interference at that position because G has no side chain (no charge, ho hydrophobic groups). Also, multiple position may have S (or similar amino acid), e.g. 613 and 616 or 613 and 614, or 614 and 616 or any combination with 617.

### Temperature Effects on Relative Incorporation Efficiencies of 3′-Phosphate-Blocked and Natural-Unblocked Nucleotides.

Table 12 shows the effect of incubation temperature on the relative incorporation efficiencies of 3′ phosphate blocked nucleotide and natural un-blocked nucleotide. The relative “spike” percentage of natural nucleotide in the reaction mix was 4% (0.12 uM) so with an equal propensity of the polymerase to incorporate natural or 3′ phosphate blocked nucleotide at The first position (P1) it may be expected that the P2 intensity would be about 4% of a potential full incorporation of natural nucleotide at P1. Here, P2 measures the ability to incorporate a reporter at the second position after the initial test nucleotide(s) were allowed to incorporate at P1 and would be expected to report the level of natural nucleotide incorporation at P1. However, the P2 incorporation percentages observed were generally higher than what may be expected from an equal incorporation efficiency of 3′ phosphate and natural nucleotide, and varied depending on the enzyme variant and the reaction temperature. SFm19r5 appeared to show the highest preference for natural nucleotide and SFr51 showed the least preference for natural nucleotide. The temperature of incubation also appeared to influence the relative incorporation efficiencies of natural versus 3′ phosphate-blocked nucleotide with SFr51 showing a decrease in P2 intensity from 18.8% at 55° C. to 10.7% at 65° C. suggesting more 3′ phosphate was incorporated at the higher temperature since a successful incorporation of blocked nucleotide at P1 should not allow the reporter to incorporate at P2. Both SFr52 and SFm19r5 also showed a similar trend in relative incorporation efficiencies with lower P2 percentages suggesting blocked nucleotide incorporation was more efficient relative to natural unblocked at higher temperatures.

An alternative explanation for the lower P2 percentage values could be that at the higher temperatures the polymerase mismatch rate increased such that after incorporation of natural nucleotide at P1 (T: A full-match) the natural nucleotide incorporated at P2 (T:G mis-match) and as a consequence blocked the ability of the P2 reporter nucleotide (ddCTP-Cy5) from incorporating. The relative decrease of P2 intensity at nearly two-fold with a 10° C. change in temperature would suggest a large shift in mismatch propensity would be needed for this to be the only explanation for the lower P2 intensity with increased temperature.

Table 2 also showed P2 intensity values after a mixed incorporation of natural and 3′ phosphate blocked nucleotide. In contrast with the short incubation reactions in solution phase (Table 12) the experiment performed for an extended time (2 minutes) on a streptavidin coated substrate Table 2) generally demonstrated lower P2 incorporation of reporter suggesting less natural nucleotide incorporation at P1. One possible explanation for this observation is an increased “forced” incorporation at the second position of the natural nucleotide (T:G mismatch) when the natural nucleotide was incorporated at P1 (T: A full match) for the extended time and higher Mn² concentration. Under such conditions the ability of the P2 reporter to incorporate is decreased and it could appear that natural nucleotide incorporation is not favored.

An increase in P1 percentage intensities was also generally observed with increasing temperature (Table 12). This could suggest that overall incorporation efficiencies have declined slightly at the higher temperature since both natural-unblocked and 3′ phosphate blocked nucleotide incorporation should suppress the reporter intensity at P1. Although it cannot be excluded that the decreased incorporation at P1 is contributing to the lower P2 values, since lower P2 can be derived from unblocked incorporation at P1, or no incorporation at P1, the relative improvement of P2 percentage values suggest that a relative shift in preference to 3′ phosphate blocked nucleotide from natural nucleotide could still be occurring at higher incubation temperatures.

TABLE 12
Percentage incorporation efficiencies of a mixture of 3 uM 3′-phosphate
blocked dTTP nucleotide and 0.12 uM un-blocked natural dTTP. Primer
and 5′ biotinylated template were hybridized in solution before the
addition of a mix of enzyme and nucleotide in reaction buffer. Nucleotide
was reacted with 0.05 ug/ul enzyme and 10 uM Mn⁺² for 20 seconds
at 55° C., 60° C., or 65° C. before the addition of EDTA to
stop the reaction. The biotinylated template with extended primer was
then allowed to bind to streptavidin coated wells of a microplate. After
washing to remove excess unbound reaction components the presence of
incorporated blocking terminator was detected by the incorporation of
labeled reporter nucleotides. 100% P1 value represents no incorporation
of the test nucleotide as determined from values obtained by incubation
without nucleotides or polymerase and 0% represents complete blocking of
the P1 position by incorporation of the test nucleotide as determined by
a primer with one additional base nucleotide occupying the P1 position.
100% P2 value represents incorporation at P2 resulting from the presence
of an un-blocked nucleobase at P1.

| Enzyme | Temperature | P1 | P2 |
|---|---|---|---|
| SFm19r5 | 55° C. | 4.9 | 95.3 |
| SFm19r5 | 60° C. | 6.7 | 80.1 |
| SFm19r5 | 65° C. | 9.7 | 79.9 |
| SFr51 | 55° C. | 3.6 | 18.8 |
| SFr51 | 60° C. | 5.6 | 13.4 |
| SFr51 | 65° C. | 6.2 | 10.7 |
| SFr52 | 55° C. | 5.8 | 25.3 |
| SFr52 | 60° C. | 4.7 | 18.1 |
| SFr52 | 65° C. | 6.1 | 14.5 |

### Example 3. Additional Variant Polymerases

Using SFr52 as a starting sequence, additional polymerase variants were generated with substitutions at the following residues: A616G (SFr85), R617F (SFr86) and R617K (SFr87) (Table 13). Table 14 demonstrates that these mutants are able to incorporate the 3P nucleotide, although they did not achieve the same level of incorporation as the SFr51 and SFr52 mutants within the allotted time of the test.

The SFr51 mutant was also used as the base sequence for additional amino acid changes S614D and S614T in SFr88 and SFr89. Again, although incorporation was demonstrated, incorporation efficiency appeared lower than that of the parent SFr51 variant. Variants SF74 (SF73 with A616L) and SF75 (SF73 with A616G) did not appear to have improved incorporation efficiency over the parent SF73 variant and in general showed poor incorporation for all bases with dGTP-3P performing the best.

TABLE 13
Amino acids of key position in the Stoffel
fragment variants of Taq polymerases.

| | Taq position | | | | | | | | | | | | | | |
|---|---|---|---|---|---|---|---|---|---|---|---|---|---|---|---|
| | 518 | 520 | 540 | 583 | 613 | 614 | 615 | 616 | 617 | 655 | 657 | 667 | 681 | 742 | 747 |
| SFr85 | V | E | K | N | Q | S | G | G | R | N | M | Y | K | N | R |
| SFr86 | V | E | K | N | Q | S | G | A | F | N | M | Y | K | N | R |
| SFr87 | V | E | K | N | Q | S | G | A | K | N | M | Y | K | N | R |
| SFr88 | V | E | K | N | Q | D | G | L | R | N | M | Y | K | N | R |
| SFr89 | V | E | K | N | Q | T | G | L | R | N | M | Y | K | N | R |
| SF74 | V | E | K | N | Q | S | G | L | R | D | L | Y | E | F | M |
| SF75 | V | E | K | N | Q | S | G | G | R | D | L | Y | E | E | M |
| SFr90 | V | E | K | N | S | S | G | L | R | N | M | Y | K | N | R |
| SFr91 | V | E | K | N | S | G | G | L | R | N | M | Y | K | N | R |
| Sfr92 | V | E | K | N | Q | G | G | S | R | N | M | Y | K | N | R |
| SFr93 | V | E | K | N | S | G | G | S | R | N | M | Y | K | N | R |
| SFr95 | V | E | K | N | Q | S | G | A | R | N | M | P | K | N | R |

TABLE 14
Incorporation efficiencies of 3′ phosphorylated nucleotides. Values represent the
percentage of incorporation of fluorescently labeled reporter nucleotides relative to a
maximum incorporation as determined by no test nucleotide inclusion. Reporter nucleotides
can incorporate at P1 when the site is un-occupied by the test nucleotide. (negative values
represent over subtraction of the estimated background level of incorporation.)

| 3′Phos | SFr85 | SFr86 | SFr87 | SFr88 | SFr89 | SFr51 | SFr52 | SF73 | SF74 | SF75 |
|---|---|---|---|---|---|---|---|---|---|---|
| dATP-3P | 20.8 | 76.6 | 16.3 | 13.3 | 52.4 | 4.4 | 5.0 | 44.8 | 65.4 | 67.6 |
| dTTP-3P | −5.3 | 39.4 | −11.9 | −5.8 | −6.4 | −8.3 | 2.7 | 60.1 | 45.5 | 35.2 |
| dGTP-3P | 5.1 | 21.1 | 9.8 | 19.4 | 21.3 | 4.6 | 3.9 | 6.6 | 12.0 | 12.6 |
| dCTP-3P | 2.8 | 21.0 | 7.3 | 3.9 | 2.6 | 3.0 | 1.4 | 111.6 | 60.1 | 38.9 |

An additional series of variants were created around positions 613-616. These included SFr90; (SFr51 with 613S), SFr91; (SFr51 with 613S and 614G), SFr92: (SFr51 with 614G, 616S), SFr93; (SFr51 with 613S, 614G, 616S). Table 15 shows that most of these variant changes were detrimental to incorporation efficiency, although variant SFr90 generally showed less decrease of incorporation efficiency relative to SFr51 with the exception of dATP-3P which appeared 6-fold less efficient. The test conditions here used a 0.5 uM concentration of nucleotides rather than the standard 3 uM.

TABLE 15
Incorporation efficiencies of 3′ phosphorylated nucleotides. Values represent the
percentage of incorporation of fluorescently labeled reporter nucleotides relative to a
maximum incorporation as determined by no test nucleotide inclusion. Reporter nucleotides
can incorporate at P1 when the site is un-occupied by the test nucleotide. (negative values
represent over subtraction of the estimated background level of incorporation.)

| 3′Phos | SFr90 | SFr91 | SFr92 | SFr93 | SFr51 | SFr52 |
|---|---|---|---|---|---|---|
| dATP-3P | 69.3 | 100.8 | 93.0 | 147.1 | 12.3 | 12.7 |
| dTTP-3P | 4.1 | 33.5 | 71.2 | 80.5 | 2.1 | 4.3 |
| dGTP-3P | 3.6 | 30.9 | 55.8 | 92.3 | 2.2 | 1.5 |
| dCTP-3P | 4.2 | 38.2 | 84.7 | 61.3 | 1.0 | 2.0 |

### Example 4. Antibody Detection of 3′ Phosphate Groups

The ability to detect small chemical modifications of nucleobases using antibodies has been reported (Drmanac, et al. bioRxiv, doi:10.1101/2020.02.19.953307, posted Feb. 20, 2020; Drmanac et al., U.S. Pat. Application Publication US20180223358). Detected modifications have included 3′ azidomethyl, and 3′ nitrobenzyl. Here, we describe the production of rabbit monoclonal antibodies that specifically recognize the terminal base of an extended DNA primer and a 3′ blocking group, in this case a phosphate moiety. Monoclonal antibodies were prepared and fluorescently labeled by NHS ester reaction to lysine amino acids within the protein IgG heavy and light chains as previously described (Drmanac et al, bioRxiv, 2020, supra).

### Selection of Antibody Clones

Clonal B-cell supernatants were screened by ELISA for a specific, positive reaction to a 3′ phosphate blocked nucleoside. A selection of clones was sequenced for the heavy and light chains of the expressed IgG and a further subset used for cellular plasmid expression, from which, purified clonal antibodies were further tested for specificity and removal from 3′phosphate incorporated nucleotides on oligonucleotide substrates. Tables 16A and 16B show predicted CDR sequences as assigned by IMGT of heavy chain variable regions (Table 16A) and light chain variable regions (Table 16 B) of selected clones.

TABLE 16A
Illustrative heavy chain variable region CDR sequences of antibodies
that bind 3′ phosphate blocked nucleosides

| V_{H} CDR sequences | CDR1 | CDR2 | CDR3 |
|---|---|---|---|
| 3P-dA | | | |
| 18B10 | GIDLSSYA | ISRRGTS (SEQ | ARATYDDYTDLNL (SEQ ID |
| | (SEQ ID NO: 3) | ID NO: 4) | NO: 5) |
| 19F1 | GIDLSSYA | ISRRGRS | ARATYDDYTDLNL (SEQ ID |
| | (SEQ ID NO: 3) | (SEQ ID NO: 6) | NO: 5) |
| 3P-dG | | | |
| 17C2 | GFSLSSFG (SEQ | IYGDTGNT | ARASYGFSRGQGFKL(SEQ |
| | ID NO: 7) | (SEQ ID NO: 8) | ID NO: 9) |
| 6B9 | GFSLGSFG | IYGDTGNT | ARTSYGGSRSQGFQL(SEQ |
| | (SEQ ID NO: 10) | (SEQ ID NO: 8) | ID NO: 11) |
| 3P-dC | | | |
| 20E9 | GFSLSAYH | ITRSAST (SEQ | VRGSIAATSTGTGL (SEQ ID |
| | (SEQ ID NO: 12) | ID NO: 13) | NO: 14) |
| 2F7 | GFSLSNYA | ISRTGSI (SEQ | ARTDYFANGDI (SEQ ID |
| | (SEQ ID NO: 15) | ID NO: 16) | NO: 17) |
| 3P-dT | | | |
| 12D6 | GIDLSSYT (SEQ | IYVHDGAT | ASGGGTTYYRI (SEQ ID |
| | ID NO: 18) | (SEQ ID NO: 19) | NO: 20) |
| 6F11 | GFSLSNYP | ISRKGTT (SEQ | ARGEYYGDILNYENYFSA |
| | (SEQ ID NO: 21) | ID NO: 22) | (SEQ ID NO: 23) |

TABLE 16B
Illustrative light chain variable region CDR sequences of antibodies
that bind 3′ phosphate blocked nucleosides

| | CDR1 | CDR2 | |
|---|---|---|---|
| 3P-dA | | | |
| 18B10 | QSVYNNNL | KTS | LGGYYSGSGWYST (SEQ ID |
| | (SEQ ID NO: 24) | | NO: 25) |
| 19F1 | QSVYDNNL | KTS | LGGYYSSSGWYST (SEQ ID |
| | (SEQ ID NO: 26) | | NO: 27) |
| 3P-dG | | | |
| 17C2 | QSVNKNNY | SAS | LGSGDFSSGDSIV (SEQ ID |
| | (SEQ ID NO: 28) | | NO: 29) |
| 6B9 | QSVNKNNY | SAS | LGSGDFSGGDSIV (SEQ ID |
| | (SEQ ID NO: 28) | | NO: 30) |
| 3P-dC | | | |
| 20E9 | QSVDNNNN | LAS | AGYSGGTTDGSA (SEQ ID |
| | (SEQ ID NO: 31) | | NO: 32) |
| 2F7 | QSINSW (SEQ | AAS | QSYDMIVNHGAP (SEQ ID |
| | ID NO: 33) | | NO: 34) |
| 3P-dT | | | |
| 12D6-L | QSVYGHNR | YTS | AGGYNGNIYA (SEQ ID |
| | (SEQ ID NO: 35) | | NO: 36) |
| 6F11-L | QSISTY (SEQ ID | RAS | QSNYYISSGNYGAV (SEQ |
| | NO: 37) | | ID NO: 38) |

3′ Phosphate Primer Detection with Antibodies in a Microtiter Plate

3′ phosphate blocked nucleotides were incorporated onto the terminal position of a primer and then reacted with unlabeled or labeled antibodies. After a short reaction time of less than 5 minutes, excess antibodies were washed from the reaction well and bound antibodies detected. In the case of unlabeled antibodies, a secondary fluorescently labeled antibody or nanobody was used to bind to the primary IgG before fluorescence detection. In the case of directly labeled primary antibodies, excess un-bound antibodies were washed away and fluorescence in the reaction well measured with a fluorescence plate reader.

Table 17 shows the fluorescence intensity obtained from antibody bound to a 3′ phosphate-extended primer compared with the same reaction conditions with no primer extension. Under some binding and wash conditions some non-specific binding of antibody was occurring on the target. However, in the presence of magnesium, the non-specific binding was dramatically reduced. The interaction of magnesium with the phosphates of the DNA phosphodiester bonds may be suppressing the ability of the antibodies to react with internal phosphates but still allowing recognition of the terminal phosphate. Specific binding in the presence of magnesium was 10-27 fold higher than without primer.

TABLE 17
Oligonucleotide template was bound to streptavidin coated reaction
wells of a 96-well microtiter plate. Primer was hybridized to the
template in some wells or buffer only was added in the no-primer
condition. A 3′ phosphate nucleotide was incorporated onto the
terminal position of the primer that was complementary to the template
strand sequence at that position. After washing to remove excess
reaction components, a single, fluorescently labeled antibody selected
from the set A-18B10, C-2F7, G-17C2, or T-12D6 was reacted with the
template/primer substrate in either a magnesium containing or magnesium
free buffer. After binding for 5 min the excess antibody was washed
at 60° C. and the plate scanned at the corresponding excitation
and emission wavelengths for the fluorescent dyes.

| | | | ratio to |
|---|---|---|---|
| | | intensity | no primer |
| A | no primer | 88385 | |
| A | primer | 265039 | 3.0 |
| A-20 mM Mg | no primer | 8820 | |
| A-20 mM Mg | primer | 238771 | 27.1 |
| G | no primer | 37695 | |
| G | primer | 94247 | 2.5 |
| G420 mM Mg | no primer | 4978 | |
| G-20 mM Mg | primer | 53159 | 10.7 |
| T | no primer | 206733 | |
| T | primer | 345210 | 1.7 |
| T-20 mMMg | no primer | 13278 | |
| T-20 mMMg | primer | 273502 | 20.6 |
| C | no primer | 19124 | |
| C | primer | 167623 | 8.8 |
| C-20 mM Mg | no primer | 11357 | |
| C-20 mM Mg | primer | 183669 | 16.2 |

3′ Phosphate Primer Detection with Antibodies on MGI-SEQ 2000

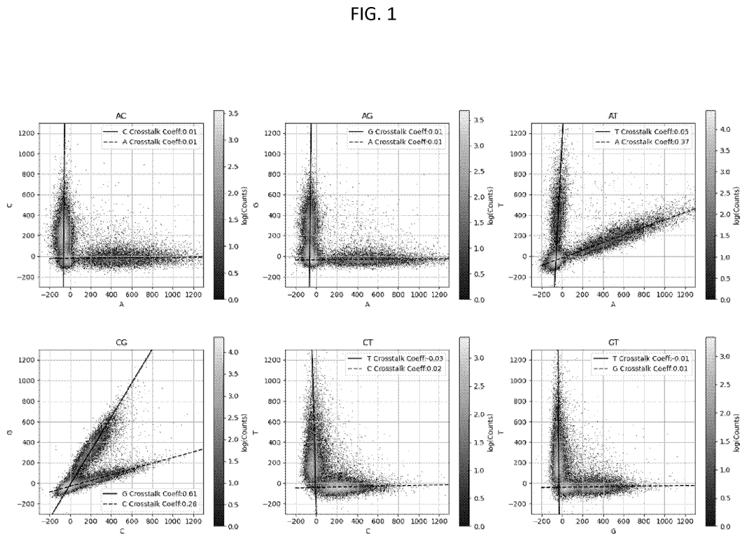

FIG. 1 shows intensity cluster plots of DNBs from one imaging cycle with four fluorescently labeled antibodies. Each spot of the clusters represents the intensity profile in two spectral wavelength channels of a single DNB. It can be seen that DNBs cluster based upon the predominant intensity of the DNB. A position away from the axes suggests intensity contribution from the alternative intensity channel which could be due to spectral cross-talk of the fluorophores, as evident in the smaller angle between the clusters in the C-G profile. Other causes of movement away from the axes could be non-specific binding of antibodies recognizing an alternative base at the terminus or a more generalized non-specific binding to elements of the DNB structure. Mixed DNBs (2 or more DNBs per binding site) could also move away from the axes. FIG. 1 demonstrates that most DNBs cluster in distinct groups that allow accurate base calling and DNB sequence determination.

### Example 5. Sequencing with 3′ Phosphate Nucleotides and Antibodies on MGI-SEQ 2000

In a similar approach as previously described for a 3′ azidomethyl blocking group (Drmanac et al, bioRxiv, 2020, supra), the 3′ phosphate blocked nucleotides were incorporated on primers hybridized to DNB nanoballs attached to a flow-cell surface. The flow cell surface was washed with buffer after incorporation of the 3′P blocked nucleotide and before binding the fluorescently labeled antibodies. After another wash step the flow cell surface was imaged using a MGI-SEQ 2000 DNB sequencer and the fluorescent intensities of DNBs were analyzed. T4 PNK was used for an incubation time of 3 min and at a concentration of 0.1 Units/μl for removal of the 3′ phosphate blocking group and regeneration of the 3′-OH for incorporation of the nucleotide for the next cycle of sequencing.

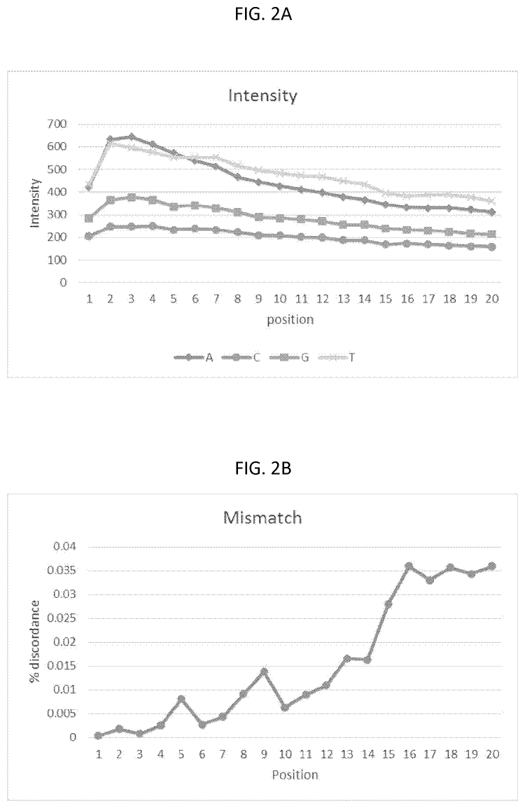

DNB intensities were then aggregated and processed to remove background and normalize the 4 different wavelength channels. The processed intensities of the DNBs are shown below in FIG. 2A. After an initial peak of intensity, in early cycles of sequencing the intensity shows a gradual decline as cycles progress. FIG. 2B shows the discordance or base-calling error estimates after mapping the 20 base reads to a human genomic reference sequence. Discordance rates increase with increasing cycles as intensity drops and out-of-phase subunits of the DNBs increase in frequency.

It is understood that the examples and embodiments described herein are for illustrative purposes only and that various modifications or changes in light thereof will be suggested to persons skilled in the art and are to be included within the spirit and purview of this application and scope of the appended claims.

All publications, patents, and patent applications cited herein are hereby incorporated by reference with respect to the material for which they are expressly cited.

### Variable Region Sequences of Illustrative Antibodies to 3′-Phosphate Nucleosides

dATP-3P antibody variable region sequences:
18B10-heavy chain variable region
(SEQ ID NO: 39)
METGLRWLLLVAVLKGVQCQSVEESGGRLVTPGTPLTLTCTVSGIDLSSYAMGWVRQAPG
KGLEWIGMISRRGTSYYASWARGRFTISKTSTTVDLKITSPTTEETATYFCARATYDDYT
DLNLWGQGTLVTVSSGQPKAPSVFPLAPCCGDTPSSTVTLGCLVKGYLPEPVTVTWNSGT
LTNGVRTFPSVRQSSGLYSLSSVVSVTSSSQPVTCNVAHPATNTKVDKTVAPSTCSKPMC
PPPELPGGPSVFIFPPKPKDTLMISRTPEVTCVVVDVSQDDPEVQFTWYINNEQVRTARP
PLREQQFNSTIRVVSTLPIAHQDWLRGKEFKCKVHNKALPAPIEKTISKARGQPLEPKVY
TMGPPREELSSRSVSLTCMINGFYPSDISVEWEKNGKAEDNYKTTPTVLDSDGSYFLYSK
LSVPTSEWQRGDVFTCSVMHEALHNHYTQKSISRSPGK*
18B10-light chain variable region
(SEQ ID NO: 40)
MDTRAPTQLLGLLLLWLPGATFAQVLTQTPSSVSAAVGGTVTINCQSSQSVYNNNLLSWY
QQKPGQPPKLLIYKTSSLASGVPSRFSGSGSGTQFTLTISGVQCDDAATYYCLGGYYSGS
GWYSTFGGGTEVVVKGDPVAPTVLIFPPSADLVATGTVTIVCVANKYFPDVTVTWEVDGT
TQTTGIENSKTPQNSADCTYNLSSTLTLTSTQYNSHKEYTCKVTQGTTSVVQSFNRGDC*
19F1-heavy chain variable region
(SEQ ID NO: 41)
METGLRWLLLVAVLKGVOQCQSVEESGGRLVTPGTPLTLTCTVSGIDLSSYAMGWVRQAPG
KGLEWIGMISRRGRSYYASWARGRFTISKTSTTVDLKITSPTTEETATYFCARATYDDYT
DLNLWGQGTLVTVSSGQPKAPSVFPLAPCCGDTPSSTVTLGCLVKGYLPEPVTVTWNSGT
LTNGVRTFPSVRQSSGLYSLSSVVSVTSSSQPVTCNVAHPATNTKVDKTVAPSTCSKPMC
PPPELPGGPSVFIFPPKPKDTLMISRTPEVTCVVVDVSQDDPEVQFTWYINNEQVRTARP
PLREQQFNSTIRVVSTLPIAHQDWLRGKEFKCKVHNKALPAPIEKTISKARGQPLEPKVY
TMGPPREELSSRSVSLTCMINGFYPSDISVEWEKNGKAEDNYKTTPTVLDSDGSYFLYSK
LSVPTSEWQRGDVFTCSVMHEALHNHYTQKSISRSPGK*
19F1-light chain variable region
(SEQ ID NO: 42)
MDTRAPTQLLGLLLLWLPGATFAQVLTQTPSSVSAAVGGTVTINCQASQSVYDNNLLSWY
QQKPGQPPKLLIYKTSSLASGVPSRFSGSGSGTQFTLTISGVQCDDAATYYCLGGYYSSS
GWYSTFGGGTEVVVKGDPVAPTVLIFPPSADLVATGTVTIVCVANKYFPDVTVTWEVDGT
TQTTGIENSKTPQNSADCTYNLSSTLTLTSTQYNSHKEYTCKVTQGTTSVVQSFNRGDC*
dCTP-3P antibody variable region sequences:
2F7-heavy chain variable region
(SEQ ID NO: 43)
METGLRWLLLVAVLKGVQCQSVEESGGRLVTPGTPLTLTCTVSGFSLSNYAIIWVRQAPG
EGLEYIGFISRTGSINYANWAKGRFTISKTSTTVDLKITSPTTEDTATYFCARTDYFANG
DIWGPGTLVTVSSGQPKAPSVFPLAPCCGDTPSSTVTLGCLVKGYLPEPVTVTWNSGTLT
NGVRTFPSVRQSSGLYSLSSVVSVTSSSQPVTCNVAHPATNTKVDKTVAPSTCSKPMCPP
PELPGGPSVFIFPPKPKDTLMISRTPEVTCVVVDVSQDDPEVQFTWYINNEQVRTARPPL
REQQFNSTIRVVSTLPIAHQDWLRGKEFKCKVHNKALPAPIEKTISKARGQPLEPKVYTM
GPPREELSSRSVSLTCMINGFYPSDISVEWEKNGKAEDNYKTTPTVLDSDGSYFLYSKLS
VPTSEWQRGDVFTCSVMHEALHNHYTQKSISRSPGK*
2F7-light chain variable region
(SEQ ID NO: 44)
MDTRVPTQLLGLLLLWLPGARCADVVMTQTPASVSEPVGGTVTIKCQASQSINSWLSWYQ
QKPGQPPKLLIYAASTLASGVPSRFKGSGSGTQFTLTISDLECADAATYFCQSYDMIVNH
GAPFGGGTGVVVSGDPVAPTVLIFPPAADQVATGTVTIVCVANKYFPDVTVTWEVDGTTQ
TTGIENSKTPQNSADCTYNLSSTLTLTSTQYNSHKEYTCKVTQGTTSVVQSFNRGDC*
20E9-heavy chain variable region
(SEQ ID NO: 45)
METGLRWLLLVAVLKGVQCQSVEESGGRLVTPGTPLTLTCTVSGFSLSAYHITWVRQAPG
KGLEWIGIITRSASTYYASWAIGRFTISKTSSTTVDLKMTSLTTEDTATYFCVRGSIAAT
STGTGLWGPGTLVTVSSGQPKAPSVFPLAPCCGDTPSSTVTLGCLVKGYLPEPVTVTWNS
GTLTNGVRTFPSVRQSSGLYSLSSVVSVTSSSQPVTCNVAHPATNTKVDKTVAPSTCSKP
MCPPPELPGGPSVFIFPPKPKDTLMISRTPEVTCVVVDVSQDDPEVQFTWYINNEQVRTA
RPPLREQQFNSTIRVVSTLPIAHQDWLRGKEFKCKVHNKALPAPIEKTISKARGQPLEPK
VYTMGPPREELSSRSVSLTCMINGFYPSDISVEWEKNGKAEDNYKTTPTVLDSDGSYFLY
SKLSVPTSEWQRGDVFTCSVMHEALHNHYTQKSISRSPGK*
20E9-light chain variable region
(SEQ ID NO: 46)
MDTRAPTQLLGLLLLWLPGATFAIVMIQTPSPVSAAVGGTVTITCQASQSVDNNNNLAWY
QQKPGQPPKLLIYLASTLASGVPSRFKGSGSGTQFTLTISDVVCDDAATYYCAGYSGGTT
DGSAFGGGTEVVVKGDPVAPTVLIFPPAADQVATGTVTIVCVANKYFPDVTVTWEVDGTT
QTTGIENSKTPQNSADCTYNLSSTLTLTSTQYNSHKEYTCKVTQGTTSVVQSFNRGDC*
dGTP-3P antibody variable region sequences:
6B9-heavy chain variable region
(SEQ ID NO: 47)
METGLRWLLLVAVLKGVQCQSLEESGGRLVTPGTPLTLTCTVSGFSLGSFGLIWVRQAPG
KGLEWIGIIYGDTGNTYYASWAKGRFTISKTSTTVDLRITSPTTEDTATYFCARTSYGGS
RSQGFQLWGQGTLVTVSSGQPKAPSVFPLAPCCGDTPSSTVTLGCLVKGYLPEPVTVTWN
SGTLTNGVRTFPSVRQSSGLYSLSSVVSVTSSSQPVTCNVAHPATNTKVDKTVAPSTCSK
PMCPPPELPGGPSVFIFPPKPKDTLMISRTPEVTCVVVDVSQDDPEVQFTWYINNEQVRT
ARPPLREQQFNSTIRVVSTLPIAHQDWLRGKEFKCKVHNKALPAPIEKTISKARGQPLEP
KVYTMGPPREELSSRSVSLTCMINGFYPSDISVEWEKNGKAEDNYKTTPTVLDSDGSYFL
YSKLSVPTSEWQRGDVFTCSVMHEALHNHYTQKSISRSPGK*
6B9-light chain variable region
(SEQ ID NO: 48)
MDTRAPTQLLGLLLLWLPGATFAQIVMTQTPASVSATVGGTVTINCQASQSVNKNNYLSW
YQQKPGQPPKLLIYSASWLASGVPSRFKGSGSGTQFTLTISDVQCDDAATYYCLGSGDES
GGDSIVFGGGTEVVVRGDPVAPTVLIFPPAADQVATGTVTIVCVANKYFPDVTVTWEVDG
TTQTTGIENSKTPQNSADCTYNLSSTLTLTSTQYNSHKEYTCKVTQGTTSVVQSFNRGDC*
17C2-heavy chain variable region
(SEQ ID NO: 49)
METGLRWLLLVAVLKGVQCQSLEESGGRLVTPGTSLTLTCTVSGFSLSSFGLIWVRQAPG
KGLEWIGIIYGDTGNTYYASWAKGRFTISKTSTTIDLRITNPTTEDTATYFCARASYGES
RGQGFKLWGQGTLVTVSSGQPKAPSVFPLAPCCGDTPSSTVTLGCLVKGYLPEPVTVTWN
SGTLTNGVRTFPSVRQSSGLYSLSSVVSVTSSSQPVTCNVAHPATNTKVDKTVAPSTCSK
PMCPPPELPGGPSVFIFPPKPKDTLMISRTPEVTCVVVDVSQDDPEVQFTWYINNEQVRT
ARPPLREQQFNSTIRVVSTLPIAHQDWLRGKEFKCKVHNKALPAPIEKTISKARGQPLEP
KVYTMGPPREELSSRSVSLTCMINGFYPSDISVEWEKNGKAEDNYKTTPTVLDSDGSYFL
YSKLSVPTSEWQRGDVFTCSVMHEALHNHYTQKSISRSPGK*
17C2-light chain variable region
(SEQ ID NO: 50)
MDTRAPTQLLGLLLLWLPGATFAQIVMTQTPASVSAAVGGTVTINCQASQSVNKNNYLSW
YQQKPGQPPKLLIYSASWLASGVPSRFSGSGSGTQFTLTISDVQCDDAATYYCLGSGDES
SGDSIVFGGGTEVVVRGDPVAPTVLIFPPAADQVATGTVTIVCVANKYFPDVTVTWEVDG
TTQTTGIENSKTPQNSADCTYNLSSTLTLTSTQYNSHKEYTCKVTQGTTSVVQSFNRGDC*
dTTP-3P antibody variable region sequences:
12D6-heavy chain variable region
(SEQ ID NO: 51)
METGLRWLLLVAVLKGVQCQSLEESGGRLVTPGTPLTLTCTVSGIDLSSYTITWVRQAPG
KGLEWIGYIYVHDGATYYANWAKGRFTISRTSSTTVDLKMTSLTTEDTATYFCASGGGTT
YYRIWGPGTLVTVSSGQPKAPSVFPLAPCCGDTPSSTVTLGCLVKGYLPEPVTVTWNSGT
LTNGVRTFPSVRQSSGLYSLSSVVSVTSSSQPVTCNVAHPATNTKVDKTVAPSTCSKPMC
PPPELPGGPSVFIFPPKPKDTLMISRTPEVTCVVVDVSQDDPEVQFTWYINNEQVRTARP
PLREQQFNSTIRVVSTLPIAHQDWLRGKEFKCKVHNKALPAPIEKTISKARGQPLEPKVY
TMGPPREELSSRSVSLTCMINGFYPSDISVEWEKNGKAEDNYKTTPTVLDSDGSYFLYSK
LSVPTSEWQRGDVFTCSVMHEALHNHYTQKSISRSPGK*
12D6-light chain variable region
(SEQ ID NO: 52)
MDTRAPTQLLGLLLLWLPGATFAQLLTQTPPSVSAPVGGTVTINCQSSQSVYGHNRLSWY
QQKPGQPPKLLIYYTSTLASGVPSRFKGSGSGTQFTLTISDLECDDAATYYCAGGYNGNI
YAFGGGTEVVVKGDPVAPTVLIFPPAADQVATGTVTIVCVANKYFPDVTVTWEVDGTTQT
TGIENSKTPQNSADCTYNLSSTLTLTSTQYNSHKEYTCKVTQGTTSVVQSFNRGDC*
6F11-heavy chain variable region
(SEQ ID NO: 53)
METGLRWLLLVAVLKGVQCQSVEESGGRLVTPGTPLTLTCTVSGFSLSNYPMNWVRQAPG
KGLEYIGIISRKGTTYYASWAKGRFTISKTSTTVDLKMTSPTIEDTGTYFCARGEYYGDI
LNYENYFSAWGPGTLVTVSSGQPKAPSVFPLAPCCGDTPSSTVTLGCLVKGYLPEPVTVT
WNSGTLTNGVRTFPSVRQSSGLYSLSSVVSVTSSSQPVTCNVAHPATNTKVDKTVAPSTC
SKPMCPPPELPGGPSVFIFPPKPKDTLMISRTPEVTCVVVDVSQDDPEVQFTWYINNEQV
RTARPPLREQQFNSTIRVVSTLPIAHQDWLRGKEFKCKVHNKALPAPIEKTISKARGQPL
EPKVYTMGPPREELSSRSVSLTCMINGFYPSDISVEWEKNGKAEDNYKTTPTVLDSDGSY
FLYSKLSVPTSEWQRGDVFTCSVMHEALHNHYTQKSISRSPGK*
6F11-light chain variable region
(SEQ ID NO: 54)
MDTRAPTQLLGLLLLWLPGARCDVVMTQTPSSVSAAVGDTVTIKCQASQSISTYLSWYQQ
KPGQPPKLLIYRASTLESGVPSRFKGSGSGTQFTLTISDLECADAATYYCQSNYYISSGN
YGAVFGGGTEVVVKGDPVAPTVLIFPPSADLVATGTVTIVCVANKYFPDVTVTWEVDGTT
QTTGIENSKTPQNSADCTYNLSSTLTLTSTQYNSHKEYTCKVTQGTTSVVQSFNRGDC*

What is claimed is:

1. An engineered DNA polymerase comprising a polypeptide sequence comprising a substitution at three, four, or five, or more of positions 610, 611, 612, 613, 614, 615, 616, and 617, as determined with reference to SEQ ID NO:1; and wherein the polypeptide sequence has at least 90% identity to SEQ ID NO:1 and incorporates a 3′-blocked nucleotide into a nucleic acid chain.

2. The engineered DNA polymerase of claim 1, wherein the polypeptide sequence comprises a substitution at three or more of positions 613, 614, 615, 616, or 617.

3. The engineered DNA polymerase of claim 1 or 2, wherein two positions are substituted with G; and a third position is substituted with S or T.

4. The engineered DNA polymerase of claim 1, wherein the polypeptide sequence comprises an A or a G at three or more of positions 613, 614, 615, 616, or 617

5. The engineered DNA polymerase of claim 1, wherein each of positions 614, 615, and 616 comprises a substitution relative to SEQ ID NO:1.

6. The engineered DNA polymerase of claim 5, wherein the substitution at position 614 is E, A, D, S, T, or G; the substitutions at position 615 is A, G, or S; or the substitution at position 616 is A, G, or S.

7. The engineered DNA polymerase of claim 6, wherein the substitution at position 614 is E, S, or T; the substitutions at position 615 is G; and the substitution at position 616 is A.

8. The engineered DNA polymerase of claim 6 or 7, wherein the substitution at position 614 is S or T.

9. The engineered DNA polymerase of claim 1, position 614 is E or S; position 615 is G or E; and position 616 is A.

10. The engineered DNA polymerase of claim 1 to 9, further comprising a substitution at position 667 as determined with reference to SEQ ID NO:1.

11. The engineered DNA polymerase of claim 10, where the substitution at position 667 is Y.

12. The engineered DNA polymerase of any one of claims 1 to 11, further comprising a substitution at one, two, three, or four of positions 655, 657, 681, 742, and 747, as determined with reference to SEQ ID NO:1.

13. The engineered DNA polymerase of any one of claims 1 to 12, further comprising a substitution at each of positions 655, 657, 681, 742, and 747, as determined with reference to SEQ ID NO:1

14. The engineered DNA polymerase of claim 12 or 13, wherein the substitution at position 655 is N; the substitution at position 657 is M; the substitution at position 681 is K; the substitution at position 742 is Q or N; or the substitution at position 747 is R.

15. The engineered DNA polymerase of claim 12 or 13, wherein the substitution at position 655 is N; the substitution at position 657 is M; the substitution at position 681 is K; the substitution at position 742 is Q or N; and the substitution at position 747 is R.

16. The engineered DNA polymerase of claim 5, comprising E, A, D, S, T, or G at position 614; G or A at position 615; A or G at position 616; and Y at position 667.

17. The engineered DNA polymerase of claim 16, further comprising an N at position 655; an M at position 657; a K at position 681; a Q or N at position 742; and a T at position 747.

18. An engineered DNA polymerase comprising a polypeptide sequence comprising substitutions at two of positions 613, 614, 615, 616, and 617; and at position 667, as determined with reference to SEQ ID NO:1; wherein the polypeptide sequence has at least 90% identity to SEQ ID NO:1 and incorporates a 3′-blocked nucleotide into a nucleic acid chain.

19. An engineered DNA polymerase of claim 18, wherein the substitution at each of the two positions is A, G, or S.

20. An engineered DNA polymerase of claim 18 or 19, wherein the polypeptide sequence comprises substitution at positions 614 and 615; as determined with reference to SEQ ID NO:1.

21. The engineered DNA polymerase of claim 18, wherein the substitution at positions 614 is E, A, D, S, T, or G; the substitution at position 615 is G or A; or the substitution at position 667 is Y.

22. The engineered DNA polymerase of claim 20, wherein the substitution at positions 614 is E, A, D, S, T, or G; the substitution at position 615 is G or A; and the substitution at position 667 is Y.

23. The engineered DNA polymerase of claim 22, were the residue at position 614 is A, S, G, or T.

24. The engineered DNA polymerase of claim 18 or 19, wherein the polypeptide sequence comprises a substitution at position 615 and 616, as determined with reference to SEQ ID NO:1.

25. The engineered DNA polymerase of claim 27, wherein the substitution at each of positions 615 and 616 is S, G, or A.

26. The engineered DNA polymerase of claim 28, wherein the substitution at each of positions 615 and 616 is G or A.

27. The engineered DNA polymerase of any one of claims 18 to 26, further comprising a substitution at each of positions 655, 657, 681, 742, and 747.

28. The engineered DNA polymerase of claim 27, wherein the substitution at position 655 is N; the substitution at position 657 is M; the substitution at position 681 is K; the substitution at position 742 is Q or N; or the substitution at position 747 is R.

29. The engineered DNA polymerase of claim 27, wherein the substitution at position 655 is N; the substitution at position 657 is M; the substitution at position 681 is K; the substitution at position 742 is Q or N; and the substitution at position 747 is R.

30. A family A polymerase comprising a set of two or more substitutions, relative to the wild-type family A polymerase sequence that together provide a larger space for accommodating a 3′-phosphate or a larger 3′-blocking group wherein the substitutions are each selected from the group consisting of A, G, S, T, and C.

31. The family A polymerase of claim 30, further comprising a Y at the position of the family A polymerase that corresponds to position 667 of Taq polymerase.

32. The family A polymerase of claim 30 or 31, comprising at least two positions in motif A in which G or A is substituted for the wildtype residue; and at least one position in motif A comprises S or T.

33. The family A polymerase of any one of claims 30 to 32, wherein a wildtype L or I residue of motif A is substituted with E, S, or T.

34. A method of incorporating a nucleotide comprising a 3′-blocking substituent into a DNA molecule, the method comprising incubating a polymerase of any one of claims 1 to 33 in a reaction mixture comprising a template nucleic acid, a primer, the nucleotide with the 3′-blocking group; and reagents to extend the primer, wherein the nucleotide comprising the 3′-blocking group is complementary to a base in the template nucleic acid and is incorporated into the DNA molecule

35. The method of claim 34, wherein the nucleotide is labeled with a detectable label.

36. A method of incorporating a first nucleotide comprising a reversible 3′-blocking substituent into a DNA molecule, the method comprising
incubating a polymerase of any one of claims 1 to 33 in a reaction mixture comprising a template nucleic acid, a primer, the nucleotide with the 3′-blocking group; and reagents to extend the primer, wherein the nucleotide comprising the 3′-blocking group is complementary to a base in the template nucleic acid and is incorporated into the extension molecule extended from the primer;
detecting the nucleotide comprising the 3′-blocking group incorporated into the extension molecule; and
removing the 3′ blocking substituent.

37. The method of claim 36, wherein the nucleotide is labeled with a detectable label.

38. The method of claim 36 or 37, further comprising incubating the extension molecule in the reaction mixture with a second nucleotide comprising a reversible blocking substituent; wherein the second nucleotide comprising the 3′-blocking group is complementary to a base in the template nucleic acid and is incorporated into the extension molecule.

39. The method of claim 36, wherein the nucleotide comprising the 3′-blocking group is detected with an affinity agent that recognizes the 3′-blocked nucleotide.

40. A kit comprising a polymerase of any one of claims 1 to 33.

41. The kit of claim 40, further comprising a 3′-blocked nucleotide.

42. A polynucleotide comprising a nucleic acid sequence encoding a polymerase of any one of claims 1 to 33.

43. A host cell comprising the polynucleotide of claim 42.


SEQUENCE LISTING (54 sequences)

SEQ ID NO: 1 - protein, 540 aa, Artificial Sequence
  Taq polymerase
ALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 2 - protein, 540 aa, Artificial Sequence
  Polypeptide sequence
ALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQSGARVLAHLSGDENLIRVFQEGRDIHTETASWMFGVPREAVNPMMRRAAKTINYGVLYGMSAHRLSQKLAIPYEEAQAFIERYFQSFPKVRAWIEKTLEEGRRRGYVETLFGRRRYVPDLEARVKSVRNAAERRAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 3 - protein, 8 aa, Artificial Sequence
  3P-dA CDR1
GIDLSSYA

SEQ ID NO: 4 - protein, 7 aa, Artificial Sequence
  3P-dA CDR2
ISRRGTS

SEQ ID NO: 5 - protein, 13 aa, Artificial Sequence
  3P-dA CDR3
ARATYDDYTDLNL

SEQ ID NO: 6 - protein, 7 aa, Artificial Sequence
  3P-dA CDR2
ISRRGRS

SEQ ID NO: 7 - protein, 8 aa, Artificial Sequence
  3P-dG CDR1
GFSLSSFG

SEQ ID NO: 8 - protein, 8 aa, Artificial Sequence
  3P-dG CDR2
IYGDTGNT

SEQ ID NO: 9 - protein, 15 aa, Artificial Sequence
  3P-dG CDR3
ARASYGFSRGQGFKL

SEQ ID NO: 10 - protein, 8 aa, Artificial Sequence
  3P-dG CDR1
GFSLGSFG

SEQ ID NO: 11 - protein, 15 aa, Artificial Sequence
  3P-dG CDR3
ARTSYGGSRSQGFQL

SEQ ID NO: 12 - protein, 8 aa, Artificial Sequence
  3P-dC CDR1
GFSLSAYH

SEQ ID NO: 13 - protein, 7 aa, Artificial Sequence
  3P-dC CDR2
ITRSAST

SEQ ID NO: 14 - protein, 14 aa, Artificial Sequence
  3P-dC CDR3
VRGSIAATSTGTGL

SEQ ID NO: 15 - protein, 8 aa, Artificial Sequence
  3P-dC CDR1
GFSLSNYA

SEQ ID NO: 16 - protein, 7 aa, Artificial Sequence
  3P-dC CDR2
ISRTGSI

SEQ ID NO: 17 - protein, 11 aa, Artificial Sequence
  3P-dC CDR3
ARTDYFANGDI

SEQ ID NO: 18 - protein, 8 aa, Artificial Sequence
  3P-dT CDR1
GIDLSSYT

SEQ ID NO: 19 - protein, 8 aa, Artificial Sequence
  3P-dT CDR2
IYVHDGAT

SEQ ID NO: 20 - protein, 11 aa, Artificial Sequence
  3P-dT CDR3
ASGGGTTYYRI

SEQ ID NO: 21 - protein, 8 aa, Artificial Sequence
  3P-dT CDR1
GFSLSNYP

SEQ ID NO: 22 - protein, 7 aa, Artificial Sequence
  3P-dT CDR2
ISRKGTT

SEQ ID NO: 23 - protein, 18 aa, Artificial Sequence
  3P-dT CDR3
ARGEYYGDILNYENYFSA

SEQ ID NO: 24 - protein, 8 aa, Artificial Sequence
  3P-dA  CDR1
QSVYNNNL

SEQ ID NO: 25 - protein, 13 aa, Artificial Sequence
  3P-dA
LGGYYSGSGWYST

SEQ ID NO: 26 - protein, 8 aa, Artificial Sequence
  3P-dA  CDR1
QSVYDNNL

SEQ ID NO: 27 - protein, 13 aa, Artificial Sequence
  3P-dA
LGGYYSSSGWYST

SEQ ID NO: 28 - protein, 8 aa, Artificial Sequence
  3P-dG CDR1
QSVNKNNY

SEQ ID NO: 29 - protein, 13 aa, Artificial Sequence
  3P-dG
LGSGDFSSGDSIV

SEQ ID NO: 30 - protein, 13 aa, Artificial Sequence
  3P-dG
LGSGDFSGGDSIV

SEQ ID NO: 31 - protein, 8 aa, Artificial Sequence
  3P-dC CDR1
QSVDNNNN

SEQ ID NO: 32 - protein, 12 aa, Artificial Sequence
  3P-dC
AGYSGGTTDGSA

SEQ ID NO: 33 - protein, 6 aa, Artificial Sequence
  3P-dC CDR1
QSINSW

SEQ ID NO: 34 - protein, 12 aa, Artificial Sequence
  3P-dC
QSYDMIVNHGAP

SEQ ID NO: 35 - protein, 8 aa, Artificial Sequence
  3P-dT CDR1
QSVYGHNR

SEQ ID NO: 36 - protein, 10 aa, Artificial Sequence
  3P-dT
AGGYNGNIYA

SEQ ID NO: 37 - protein, 6 aa, Artificial Sequence
  3P-dT CDR1
QSISTY

SEQ ID NO: 38 - protein, 14 aa, Artificial Sequence
  3P-dT
QSNYYISSGNYGAV

SEQ ID NO: 39 - protein, 458 aa, Artificial Sequence
  dATP-3P
METGLRWLLLVAVLKGVQCQSVEESGGRLVTPGTPLTLTCTVSGIDLSSYAMGWVRQAPGKGLEWIGMISRRGTSYYASWARGRFTISKTSTTVDLKITSPTTEETATYFCARATYDDYTDLNLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 40 - protein, 239 aa, Artificial Sequence
  dATP-3P
MDTRAPTQLLGLLLLWLPGATFAQVLTQTPSSVSAAVGGTVTINCQSSQSVYNNNLLSWYQQKPGQPPKLLIYKTSSLASGVPSRFSGSGSGTQFTLTISGVQCDDAATYYCLGGYYSGSGWYSTFGGGTEVVVKGDPVAPTVLIFPPSADLVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 41 - protein, 458 aa, Artificial Sequence
  dATP-3P
METGLRWLLLVAVLKGVQCQSVEESGGRLVTPGTPLTLTCTVSGIDLSSYAMGWVRQAPGKGLEWIGMISRRGRSYYASWARGRFTISKTSTTVDLKITSPTTEETATYFCARATYDDYTDLNLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 42 - protein, 239 aa, Artificial Sequence
  dATP-3P
MDTRAPTQLLGLLLLWLPGATFAQVLTQTPSSVSAAVGGTVTINCQASQSVYDNNLLSWYQQKPGQPPKLLIYKTSSLASGVPSRFSGSGSGTQFTLTISGVQCDDAATYYCLGGYYSSSGWYSTFGGGTEVVVKGDPVAPTVLIFPPSADLVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 43 - protein, 456 aa, Artificial Sequence
  dCTP-3P
METGLRWLLLVAVLKGVQCQSVEESGGRLVTPGTPLTLTCTVSGFSLSNYAIIWVRQAPGEGLEYIGFISRTGSINYANWAKGRFTISKTSTTVDLKITSPTTEDTATYFCARTDYFANGDIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 44 - protein, 237 aa, Artificial Sequence
  dCTP-3P
MDTRVPTQLLGLLLLWLPGARCADVVMTQTPASVSEPVGGTVTIKCQASQSINSWLSWYQQKPGQPPKLLIYAASTLASGVPSRFKGSGSGTQFTLTISDLECADAATYFCQSYDMIVNHGAPFGGGTGVVVSGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 45 - protein, 460 aa, Artificial Sequence
  dCTP-3P
METGLRWLLLVAVLKGVQCQSVEESGGRLVTPGTPLTLTCTVSGFSLSAYHITWVRQAPGKGLEWIGIITRSASTYYASWAIGRFTISKTSSTTVDLKMTSLTTEDTATYFCVRGSIAATSTGTGLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 46 - protein, 238 aa, Artificial Sequence
  dCTP-3P
MDTRAPTQLLGLLLLWLPGATFAIVMIQTPSPVSAAVGGTVTITCQASQSVDNNNNLAWYQQKPGQPPKLLIYLASTLASGVPSRFKGSGSGTQFTLTISDVVCDDAATYYCAGYSGGTTDGSAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 47 - protein, 461 aa, Artificial Sequence
  dGTP-3P
METGLRWLLLVAVLKGVQCQSLEESGGRLVTPGTPLTLTCTVSGFSLGSFGLIWVRQAPGKGLEWIGIIYGDTGNTYYASWAKGRFTISKTSTTVDLRITSPTTEDTATYFCARTSYGGSRSQGFQLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 48 - protein, 240 aa, Artificial Sequence
  dGTP-3P
MDTRAPTQLLGLLLLWLPGATFAQIVMTQTPASVSATVGGTVTINCQASQSVNKNNYLSWYQQKPGQPPKLLIYSASWLASGVPSRFKGSGSGTQFTLTISDVQCDDAATYYCLGSGDFSGGDSIVFGGGTEVVVRGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 49 - protein, 461 aa, Artificial Sequence
  dGTP-3P
METGLRWLLLVAVLKGVQCQSLEESGGRLVTPGTSLTLTCTVSGFSLSSFGLIWVRQAPGKGLEWIGIIYGDTGNTYYASWAKGRFTISKTSTTIDLRITNPTTEDTATYFCARASYGFSRGQGFKLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 50 - protein, 240 aa, Artificial Sequence
  dGTP-3P
MDTRAPTQLLGLLLLWLPGATFAQIVMTQTPASVSAAVGGTVTINCQASQSVNKNNYLSWYQQKPGQPPKLLIYSASWLASGVPSRFSGSGSGTQFTLTISDVQCDDAATYYCLGSGDFSSGDSIVFGGGTEVVVRGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 51 - protein, 458 aa, Artificial Sequence
  dTTP-3P
METGLRWLLLVAVLKGVQCQSLEESGGRLVTPGTPLTLTCTVSGIDLSSYTITWVRQAPGKGLEWIGYIYVHDGATYYANWAKGRFTISRTSSTTVDLKMTSLTTEDTATYFCASGGGTTYYRIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 52 - protein, 236 aa, Artificial Sequence
  dTTP-3P
MDTRAPTQLLGLLLLWLPGATFAQLLTQTPPSVSAPVGGTVTINCQSSQSVYGHNRLSWYQQKPGQPPKLLIYYTSTLASGVPSRFKGSGSGTQFTLTISDLECDDAATYYCAGGYNGNIYAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 53 - protein, 463 aa, Artificial Sequence
  dTTP-3P
METGLRWLLLVAVLKGVQCQSVEESGGRLVTPGTPLTLTCTVSGFSLSNYPMNWVRQAPGKGLEYIGIISRKGTTYYASWAKGRFTISKTSTTVDLKMTSPTIEDTGTYFCARGEYYGDILNYENYFSAWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 54 - protein, 238 aa, Artificial Sequence
  dTTP-3P
MDTRAPTQLLGLLLLWLPGARCDVVMTQTPSSVSAAVGDTVTIKCQASQSISTYLSWYQQKPGQPPKLLIYRASTLESGVPSRFKGSGSGTQFTLTISDLECADAATYYCQSNYYISSGNYGAVFGGGTEVVVKGDPVAPTVLIFPPSADLVATGTVTIVCVANKYFPDVTVTWEVDGTTQTTGIENSKTPQNSADCTYNLSSTLTLTSTQYNSHKEYTCKVTQGTTSVVQSFNRGDC